SEQUENCE LISTING (53 sequences)

SEQ ID NO: 1 - DNA, 5574 bp, Coccidioides spp.
atggcggcggccccgtcccatgcccagtcgtctctcccctcgctgccccatcacctgcagtccgacactcaactgacggcccaccttgccagccgattccatgtctccctccccacagctcgcctttcttcgcatgctctgatatgcctgaatacgtatacatcctcaacaaaaggtcctgatggactcaaggaaggaagcgccatgggggaggcggaggatttggcaaggcgagcctggacaagattggggtcaaggggggaaaatcaagctgttgttttcctaggtgaatctggctcaggcaaaacaactattcgatctcacctgttatcttcgctcctctcattcacctcaaccccgctatctaccaagttgtcccttgcggcatttgtttttgacacactgacaactacaaaagccgttactacgccaactgcgtcaaaagccggtctctttttcgagctacaatatgatgcatcgtccaccgtaaatccgactttgattggtgggaagcttttagatcaccggctggagcggagcagaatagcgtctgttcctacgggagagcgaagttttcatgttttgtattaccttctggcggggaccagtccagccgaaaagacacatctaggtctttctagttcaggcgatgttatctcgagtggcccaaccaagcgatcttcatcccaagcgtcacacaagcgctggcgatatctcggacacccgacccaattaaaggtcggaatcaacgatgcggaaggattccagcactttaagacggcattgcggaaactggaattcccccgaagcgagatcgcagaaatttgccaaatcctggcttgtatcctgcatatcggacaacttgaattcaccacaggtcaggctacgaggacaggggcggatgaaagtggtggatattcgcacgagggcggtgagaccatcaccgtcgtcaaaaacaaggatgttctaagtattgttgccgccttccttggtctgagtgtcgatgaacttgaaacaagtttgggatataagacgaagacaatccataaagaacgggtaaccgttatgcttgatccccagggcgcccgaacaaacgcagatgggtttgcaaggacactctatgctcttctagttgcctatgtgattgaaaacatcaatcagcggatctgcgctgccgaggacgccgtggctaacaccatttccgttgttgatttccctggttttgcccagatgtctacgacaaattcgaccttggatcaattgctgaacaatgctgctaccgagtccctttatcagtattgtttgcaaaacttctttgagcgtcaggcagatatgttggaaacagaagaggtcaacgtcccagcaaccagctattttgacaactccgatgccgttaagggccttttgaaacacggcaacggtctcctctccattttggacgatcaaaccaagcgcgcccgaaccgacttgcagttcctcgagagtattcgcaaacggttcgagaataaaaacccagctatagctgttggtagctcgaccgcaattctcccaggcagcaactttgctacgcaaaacgctgccgctgtattcacagtgaggcattttgcaggggaagttgattaccccgttaagggcctaattgaagaaaatggtgatctggtatctggcgacctccttaacctcattagctctacgaagagtggatttgttcgggaactgttcggtcaagaagcattgcaaacgatccaccatcccaaggaaaggaatgctatcatgcaagctcaagtcagctcgaaaccattgagaatgccgagcatggcacggcggaaaatgagcagaccctcaaggtttacgtcttttggatctcagtccgaagctgacgacaccgatgactttgctagtacatctaccagtacgaagaagggcggatctaacagtaagaaagggtcagtggctggtgctccaaaacagggcgcagctggccagttcctcgcctctcttgacaatataaccaaatccttatcagcgtcaaatgtgaacccttattttgtcttctgccttaagccaaatgacaggcggattgcaaatcagtttgatagcaaatgcgtccggacccaagtgcagacgttggggattgccgagatcagccaacgtctccgcaatgcggacttctctgtattcttaccatttgctgaattccttgggcttgcagaagcagaatcgatcattgttgggagtgacaaggaaaagtcgcagctcatcgttgatgagaagaggtggccaaccaacgaggcacgcgttggcagcactggtgtcttcctgagtgaaagatgctgggcagatctggccaaagttggcgagcgagttcttccaagttttagtggagaaggtactgatgatggcagagatggccttctccatgttggcggcaaccaatatggagattcaaaagtccgtctcttgccttctggagactccacccctggagcgtttatatacggggatgataagcaaggatacttcggcagccgggaattggatgcacggtcggatgctggcgcatctgcctttaactcaggtgatatgttcaagaatttggaaacccgtgagcaaatggcagagcgaagcaacgagaaagagatggttgaagtggaggacgtcatcgtctcgggaagccgcaaacgctggcttgcgctcgtttatctgttgaccttttatatccctgatttcttgatcaagtgggtcgggagaaagaagagaaaggatgtgcgtgtcgcttggcgtgaaaaattcgctatcaaccttcttatatggttgagctgtggctttgtggtatttttcattatccttttcccgcgattgatctgtccgacgcaacacgttttctccgctgaagaactttcgtctcacgatgggaaaggcaaccataacgcatataccgccattcgaggagaagtatttgacttgggctcttttatacctgcccattacccgggtattgttcctcgatcggcgctgataaagtatgctggaattgatgctacgcatttattccctgtgcaggtttctgcgctttgtcggggtaaaggcggctcacaacggcccattgatccttcagtgctgttcgacttcactcccaccaatatatcgggttctgcgaacgttgttagcggaaacgacaaaaatttcctgtaccatgacttcagacacttcagaaatgatactcgaccaacctggtactttcagcaaatgcgatacctacgggcaaactacaaaaaaggagagattggctacactaagagccatctcaagacgcttgctgagaaggagtctcagtacatcggagtcataaatggagtggtttatgacttcactgactacatgattggaggtcgaattactagagacaaagatggtaaaccgaaaccaggtgtcgacgtggatttcatggattcccgagttgtcgatttgttccagcaaaaatccgggcatgatctgaccgagtacttcaacagacttcccctcagcagcacactgcgttacgatatggaagtctgcctcaacaacctcttcaaagtcggagccctagacacccgaaattcgacgcagtgcctcttctctcaatattttgtgcttgcgtgttcggctctcctggcgagtatcattggttttaagttcttggctgcattgcagtttggaagaaagaatgtcccagaaaaccttgataagtttatcatttgccaggtaccagcgtatacagaagatgaggagtctctccgtcgtgcgattgactcgatggcccggatgaaatacgatgataaacggaaactcctcgtggttatttgcgacggtatgatcattggacagggcaatgaccgccccacgcctcgaatcgtcctcgatatccttggtgtcccagagactgttgaccccgaaccactcagctttgaaagtttaggagaaggcatgaggcagcacaacatgggcaaggtttactccggactgtacgaagtgcaaggtcacattgttccatttttggtagtggttaaagttggcaagccgtctgaggtttctcgacctggtaaccgcggaaaacgtgattctcaaatgcttctgatgcgcttcctcaaccgcgtccattacaacgcaccgatgagcccgcttgaactggaaatgcatcatcaggtccgcaatattattggtgtaaatccgacattctacgagttcatcttacaggttgatgcggataccgttgttgcgcaagattctgctactcgaatggtcgcctcattcttgcatgatacgagagtgattgcactctgcggagagacagctcttaacaacgccaaatcttccatgatcacgatgattcaagtctacgaatactggatctcccacaacttgacgaaagccttcgaaagtctttttggctctgtgacctgtttaccaggttgtttcagtatgtaccgcatccgtgccgccgatacaggcaagccactcttcgtcagcaaggaagttgtagatgcctatggagagattcgtgttgatacgcttcacatgaaaaacttgctccatctgggagaggatcgatatctgacaaccctacttctgaagcaccatccaaaatataagaccaaatatattttcaatgcgcatgcatggactgttgctcctgacagctgggccgtcttcttgtcccaacgtcgcagatggatcaattcgactgtccacaacttgatcgagctcattcctctccaacaactgtgcgggttctgctgcttcagtatgaggtttgttgtttttgtggatcttctcagtacaatcattcaacctgtgaccgtcgcctatattgtctacctcattgtactcctggctatgaacaccggcatcattccaatcacagcattcattttgctcggagcgatctacggtctgcaggctattattttcattctccgccgcaaatgggaaatggtaggatggatgataatttatctccttgccatgcctgtgttctcgttgggtctgcctctctatgctttttggcatatggatgacttcacatggggtaatactcgtattgtcactggtgagaaaggccgtaaggtcgttatcacggatgagggaaagtttgatccagcttcaattcctaagaagaagtgggaggagtatcaagctgagctctgggaagcccagacgcatcgtgatgaccagtcggaagtctctggatactcctatcgaacaaagtcgtaccacccagcaatgtcggaatatggatatcccgccacaagaccagcttctcacctggattacaaccgccattcgcggctatctctgcccacttctgaactcctcggtcgtggctcagacctcgaaatgactgacttcgccggtcttccgaccgacgatgcgttgctagccgagatcagagacattctgagaacggctgacttgatgaccgttacgaagaagagcgtcaaacaagaacttgaacggagatttaacatcccgctggacgcaaaacgagcgtacattaattctgcgactgaagcagttctttcaggccaactctaa

SEQ ID NO: 2 - protein, 1857 aa, Coccidioides spp.
MAAAPSHAQSSLPSLPHHLQSDTQLTAHLASRFHVSLPTARLSSHALICLNTYTSSTKGPDGLKEGSAMGEAEDLARRAWTRLGSRGENQAVVFLGESGSGKTTIRSHLLSSLLSFTSTPLSTKLSLAAFVFDTLTTTKAVTTPTASKAGLFFELQYDASSTVNPTLIGGKLLDHRLERSRIASVPTGERSFHVLYYLLAGTSPAEKTHLGLSSSGDVISSGPTKRSSSQASHKRWRYLGHPTQLKVGINDAEGFQHFKTALRKLEFPRSEIAEICQILACILHIGQLEFTTGQATRTGADESGGYSHEGGETITVVKNKDVLSIVAAFLGLSVDELETSLGYKTKTIHKERVTVMLDPQGARTNADGFARTLYALLVAYVIENINQRICAAEDAVANTISVVDFPGFAQMSTTNSTLDQLLNNAATESLYQYCLQNFFERQADMLETEEVNVPATSYFDNSDAVKGLLKHGNGLLSILDDQTKRARTDLQFLESIRKRFENKNPAIAVGSSTAILPGSNFATQNAAAVFTVRHFAGEVDYPVKGLIEENGDLVSGDLLNLISSTKSGFVRELFGQEALQTIHHPKERNAIMQAQVSSKPLRMPSMARRKMSRPSRFTSFGSQSEADDTDDFASTSTSTKKGGSNSKKGSVAGAPKQGAAGQFLASLDNITKSLSASNVNPYFVFCLKPNDRRIANQFDSKCVRTQVQTLGIAEISQRLRNADFSVFLPFAEFLGLAEAESIIVGSDKEKSQLIVDEKRWPTNEARVGSTGVFLSERCWADLAKVGERVLPSFSGEGTDDGRDGLLHVGGNQYGDSKVRLLPSGDSTPGAFIYGDDKQGYFGSRELDARSDAGASAFNSGDMFKNLETREQMAERSNEKEMVEVEDVIVSGSRKRWLALVYLLTFYIPDFLIKWVGRKKRKDVRVAWREKFAINLLIWLSCGFVVFFIILFPRLICPTQHVFSAEELSSHDGKGNHNAYTAIRGEVFDLGSFIPAHYPGIVPRSALIKYAGIDATHLFPVQVSALCRGKGGSQRPIDPSVLFDFTPTNISGSANVVSGNDKNFLYHDFRHFRNDTRPTWYFQQMRYLRANYKKGEIGYTKSHLKTLAEKESQYIGVINGVVYDFTDYMIGGRITRDKDGKPKPGVDVDFMDSRVVDLFQQKSGHDLTEYFNRLPLSSTLRYDMEVCLNNLFKVGALDTRNSTQCLFSQYFVLACSALLASIIGFKFLAALQFGRKNVPENLDKFIICQVPAYTEDEESLRRAIDSMARMKYDDKRKLLVVICDGMIIGQGNDRPTPRIVLDILGVPETVDPEPLSFESLGEGMRQHNMGKVYSGLYEVQGHIVPFLVVVKVGKPSEVSRPGNRGKRDSQMLLMRFLNRVHYNAPMSPLELEMHHQVRNIIGVNPTFYEFILQVDADTVVAQDSATRMVASFLHDTRVIALCGETALNNAKSSMITMIQVYEYWISHNLTKAFESLFGSVTCLPGCFSMYRIRAADTGKPLFVSKEVVDAYGEIRVDTLHMKNLLHLGEDRYLTTLLLKHHPKYKTKYIFNAHAWTVAPDSWAVFLSQRRRWINSTVHNLIELIPLQQLCGFCCFSMRFVVFVDLLSTIIQPVTVAYIVYLIVLLAMNTGIIPITAFILLGAIYGLQAIIFILRRKWEMVGWMIIYLLAMPVFSLGLPLYAFWHMDDFTWGNTRIVTGEKGRKVVITDEGKFDPASIPKKKWEEYQAELWEAQTHRDDQSEVSGYSYRTKSYHPAMSEYGYPATRPASHLDYNRHSRLSLPTSELLGRGSDLEMTDFAGLPTDDALLAEIRDILRTADLMTVTKKSVKQELERRFNIPLDAKRAYINSATEAVLSGQL

SEQ ID NO: 3 - DNA, 4236 bp, Coccidioides spp.
tctacgacaaattcgaccttggatcaattgctgaacaatgctgctaccgagtccctttatcagtattgtttgcaaaacttctttgagcgtcaggcagatatgttggaaacagaagaggtcaacgtcccagcaaccagctattttgacaactccgatgccgttaagggccttttgaaacacggcaacggtctcctctccattttggacgatcaaaccaagcgcgcccgaaccgacttgcagttcctcgagagtattcgcaaacggttcgagaataaaaacccagctatagctgttggtagctcgaccgcaattctcccaggcagcaactttgctacgcaaaacgctgccgctgtattcacagtgaggcattttgcaggggaagttgattaccccgttaagggcctaattgaagaaaatggtgatctggtatctggcgacctccttaacctcattagctctacgaagagtggatttgttcgggaactgttcggtcaagaagcattgcaaacgatccaccatcccaaggaaaggaatgctatcatgcaagctcaagtcagctcgaaaccattgagaatgccgagcatggcacggcggaaaatgagcagaccctcaaggtttacgtcttttggatctcagtccgaagctgacgacaccgatgactttgctagtacatctaccagtacgaagaagggcggatctaacagtaagaaagggtcagtggctggtgctccaaaacagggcgcagctggccagttcctcgcctctcttgacaatataaccaaatccttatcagcgtcaaatgtgaacccttattttgtcttctgccttaagccaaatgacaggcggattgcaaatcagtttgatagcaaatgcgtccggacccaagtgcagacgttggggattgccgagatcagccaacgtctccgcaatgcggacttctctgtattcttaccatttgctgaattccttgggcttgcagaagcagaatcgatcattgttgggagtgacaaggaaaagtcgcagctcatcgttgatgagaagaggtggccaaccaacgaggcacgcgttggcagcactggtgtcttcctgagtgaaagatgctgggcagatctggccaaagttggcgagcgagttcttccaagttttagtggagaaggtactgatgatggcagagatggccttctccatgttggcggcaaccaatatggagattcaaaagtccgtctcttgccttctggagactccacccctggagcgtttatatacggggatgataagcaaggatacttcggcagccgggaattggatgcacggtcggatgctggcgcatctgcctttaactcaggtgatatgttcaagaatttggaaacccgtgagcaaatggcagagcgaagcaacgagaaagagatggttgaagtggaggacgtcatcgtctcgggaagccgcaaacgctggcttgcgctcgtttatctgttgaccttttatatccctgatttcttgatcaagtgggtcgggagaaagaagagaaaggatgtgcgtgtcgcttggcgtgaaaaattcgctatcaaccttcttatatggttgagctgtggctttgtggtatttttcattatccttttcccgcgattgatctgtccgacgcaacacgttttctccgctgaagaactttcgtctcacgatgggaaaggcaaccataacgcatataccgccattcgaggagaagtatttgacttgggctcttttatacctgcccattacccgggtattgttcctcgatcggcgctgataaagtatgctggaattgatgctacgcatttattccctgtgcaggtttctgcgctttgtcggggtaaaggcggctcacaacggcccattgatccttcagtgctgttcgacttcactcccaccaatatatcgggttctgcgaacgttgttagcggaaacgacaaaaatttcctgtaccatgacttcagacacttcagaaatgatactcgaccaacctggtactttcagcaaatgcgatacctacgggcaaactacaaaaaaggagagattggctacactaagagccatctcaagacgcttgctgagaaggagtctcagtacatcggagtcataaatggagtggtttatgacttcactgactacatgattggaggtcgaattactagagacaaagatggtaaaccgaaaccaggtgtcgacgtggatttcatggattcccgagttgtcgatttgttccagcaaaaatccgggcatgatctgaccgagtacttcaacagacttcccctcagcagcacactgcgttacgatatggaagtctgcctcaacaacctcttcaaagtcggagccctagacacccgaaattcgacgcagtgcctcttctctcaatattttgtgcttgcgtgttcggctctcctggcgagtatcattggttttaagttcttggctgcattgcagtttggaagaaagaatgtcccagaaaaccttgataagtttatcatttgccaggtaccagcgtatacagaagatgaggagtctctccgtcgtgcgattgactcgatggcccggatgaaatacgatgataaacggaaactcctcgtggttatttgcgacggtatgatcattggacagggcaatgaccgccccacgcctcgaatcgtcctcgatatccttggtgtcccagagactgttgaccccgaaccactcagctttgaaagtttaggagaaggcatgaggcagcacaacatgggcaaggtttactccggactgtacgaagtgcaaggtcacattgttccatttttggtagtggttaaagttggcaagccgtctgaggtttctcgacctggtaaccgcggaaaacgtgattctcaaatgcttctgatgcgcttcctcaaccgcgtccattacaacgcaccgatgagcccgcttgaactggaaatgcatcatcaggtccgcaatattattggtgtaaatccgacattctacgagttcatcttacaggttgatgcggataccgttgttgcgcaagattctgctactcgaatggtcgcctcattcttgcatgatacgagagtgattgcactctgcggagagacagctcttaacaacgccaaatcttccatgatcacgatgattcaagtctacgaatactggatctcccacaacttgacgaaagccttcgaaagtctttttggctctgtgacctgtttaccaggttgtttcagtatgtaccgcatccgtgccgccgatacaggcaagccactcttcgtcagcaaggaagttgtagatgcctatggagagattcgtgttgatacgcttcacatgaaaaacttgctccatctgggagaggatcgatatctgacaaccctacttctgaagcaccatccaaaatataagaccaaatatattttcaatgcgcatgcatggactgttgctcctgacagctgggccgtcttcttgtcccaacgtcgcagatggatcaattcgactgtccacaacttgatcgagctcattcctctccaacaactgtgcgggttctgctgcttcagtatgaggtttgttgtttttgtggatcttctcagtacaatcattcaacctgtgaccgtcgcctatattgtctacctcattgtactcctggctatgaacaccggcatcattccaatcacagcattcattttgctcggagcgatctacggtctgcaggctattattttcattctccgccgcaaatgggaaatggtaggatggatgataatttatctccttgccatgcctgtgttctcgttgggtctgcctctctatgctttttggcatatggatgacttcacatggggtaatactcgtattgtcactggtgagaaaggccgtaaggtcgttatcacggatgagggaaagtttgatccagcttcaattcctaagaagaagtgggaggagtatcaagctgagctctgggaagcccagacgcatcgtgatgaccagtcggaagtctctggatactcctatcgaacaaagtcgtaccacccagcaatgtcggaatatggatatcccgccacaagaccagcttctcacctggattacaaccgccattcgcggctatctctgcccacttctgaactcctcggtcgtggctcagacctcgaaatgactgacttcgccggtcttccgaccgacgatgcgttgctagccgagatcagagacattctgagaacggctgacttgatgaccgttacgaag

SEQ ID NO: 4 - DNA, 6734 bp, Coccidioides spp.
  misc_feature 4935, 5143, 5234: n = A,T,C or G
ctagtaacggccgccagtgtgctggaattcgcccttctacgacaaattcgaccttggatcaattgctgaacaatgctgctaccgagtccctttatcagtattgtttgcaaaacttctttgagcgtcaggcagatatgttggaaacagaagaggtcaacgtcccagcaaccagctattttgacaactccgatgccgttaagggccttttgaaacacggcaacggtctcctctccattttggacgatcaaaccaagcgcgcccgaaccgacttgcagttcctcgagagtattcgcaaacggttcgagaataaaaacccagctatagctgttggtagctcgaccgcaattctcccaggcagcaactttgctacgcaaaacgctgccgctgtattcacagtgaggcattttgcaggggaagttgattaccccgttaagggcctaattgaagaaaatggtgatctggtatctggcgacctccttaacctcattagctctacgaagagtggatttgttcgggaactgttcggtcaagaagcattgcaaacgatccaccatcccaaggaaaggaatgctatcatgcaagctcaagtcagctcgaaaccattgagaatgccgagcatggcacggcggaaaatgagcagaccctcaaggtttacgtcttttggatctcagtccgaagctgacgacaccgatgactttgctagtacatctaccagtacgaagaaggccggatctaacagtaagaaagggtcagtggctggtgctccaaaacagggcgcagctggccagttcctcgcctctcttgacaatataaccaaatccttatcagcgtcaaatgtgaacccttattttgtcttctgccttaagccaaatgacaggcggattgcaaatcagtttgatagcaaatgcgtccggacccaagtgcagacgttggggattgccgagatcagccaacgtctccgcaatgcggacttctctgtattcttaccatttgctgaattccttgggcttgcagaagcagaatcgatcattgttgggagtgacaaggaaaagtcgcagctcatcgttgatgagaagaggtggccaaccaacgaggcacgcgttggcagcactggtgtcttcctgagtgaaagatgctgggcagatctggccaaagttggcgagcgagttcttccaagttttagtggagaaggtactgatgatggcagagatggccttctccatgttggcggcaaccaatatggagattcaaaagtccgtctcttgccttctggagactccacccctggagcgtttatatacggggatgataagcaaggatacttcggcagccgggaattggatgcacggtcggatgctggcgcatctgcctttaactcaggtgatatgttcaagaatttggaaacccgtgagcaaatggcagagcgaagcaacgagaaagagatggttgaagtggaggacgtcatcgtctcgggaagccgcaaacgctggcttgcgctcgtttatctgttgaccttttatatccctgatttcttgatcaagtgggtcgggagaaagaagagaaaggatgtgcgtgtcgcttggcgtgaaaaattcgctatcaaccttcttatatggttgagctgtggctttgtggtatttttcattatccttttcccgcgattgatctgtccgacgcaacacgttttctccgctgaagaactttcgtctcacgatgggaaaggcaaccataacgcatataccgccattcgaggagaagtatttgacttgggctcttttatacctgcccattaccccctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaagtcctcgtgtactgtgtaagcgcccactccacatctccactcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggttcgaaagtctttttggctctgtgacctgtttaccaggttgtttcagtatgtaccgcatccgtgccgccgatacaggcaagccactcttcgtcagcaaggaagttgtagatgcctatggagagattcgtgttgatacgcttcacatgaaaaacttgctccatctgggagaggatcgatatctgacaaccctacttctgaagcaccatccaaaatataagaccaaatatattttcaatgcgcatgcatggactgttgctcctgacagctgggccgtcttcttgtcccaacgtcgcagatggatcaattcgactgtccacaacttgatcgagctcattcctctccaacaactgtgcgggttctgctgcttcagtatgaggtttgttgtttttgtggatcttctcagtacaatcattcaacctgtgaccgtcgcctatattgtctacctcattgtactcctggctatgaacaccggcatcattccaatcacagcattcattttgctcggagcgatctacggtctgcaggctattattttcattctccgccgcaaatgggaaatggtaggatggatgataatttatctccttgccatgcctgtgttctcgttgggtctgcctctctatgctttttggcatatggatgacttcacatggggtaatactcgtattgtcactggtgagaaaggccgtaaggtcgttatcacggatgagggaaagtttgatccagcttcaattcctaagaagaagtgggaggagtatcaagctgagctctgggaagcccagacgcatcgtgatgaccagtcggaagtctctggatactcctatcgaacaaagtcgtaccacccagcaatgtcggaatatggatatcccgccacaagaccagcttctcacctggattacaaccgccattcgcggctatctctgcccacttctgaactcctcggtcgtggctcagacctcgaaatgactgacttcgccggtcttccgaccgacgatgcgttgctagccgagatcagagacattctgagaacggctgacttgatgaccgttacgaagagggcgaattctgcagatatccatcacactggcggccgctcgagcatgcatctagagggcc

SEQ ID NO: 5 - DNA, 1420 bp, Coccidioides spp.
gggtattgttcctcgatcggcgctgataaagtatgctggaattgatgctacgcatttattccctgtgcaggtttctgcgctttgtcggggtaaaggcggctcacaacggcccattgatccttcagtgctgttcgacttcactcccaccaatatatcgggttctgcgaacgttgttagcggaaacgacaaaaatttcctgtaccatgacttcagacacttcagaaatgatactcgaccaacctggtactttcagcaaatgcgatacctacgggcaaactacaaaaaaggagagattggctacactaagagccatctcaagacgcttgctgagaaggagtctcagtacatcggagtcataaatggagtggtttatgacttcactgactacatgattggaggtcgaattactagagacaaagatggtaaaccgaaaccaggtgtcgacgtggatttcatggattcccgagttgtcgatttgttccagcaaaaatccgggcatgatctgaccgagtacttcaacagacttcccctcagcagcacactgcgttacgatatggaagtctgcctcaacaacctcttcaaagtcggagccctagacacccgaaattcgacgcagtgcctcttctctcaatattttgtgcttgcgtgttcggctctcctggcgagtatcattggttttaagttcttggctgcattgcagtttggaagaaagaatgtcccagaaaaccttgataagtttatcatttgccaggtaccagcgtatacagaagatgaggagtctctccgtcgtgcgattgactcgatggcccggatgaaatacgatgataaacggaaactcctcgtggttatttgcgacggtatgatcattggacagggcaatgaccgccccacgcctcgaatcgtcctcgatatccttggtgtcccagagactgttgaccccgaaccactcagctttgaaagtttaggagaaggcatgaggcagcacaacatgggcaaggtttactccggactgtacgaagtgcaaggtcacattgttccatttttggtagtggttaaagttggcaagccgtctgaggtttctcgacctggtaaccgcggaaaacgtgattctcaaatgcttctgatgcgcttcctcaaccgcgtccattacaacgcaccgatgagcccgcttgaactggaaatgcatcatcaggtccgcaatattattggtgtaaatccgacattctacgagttcatcttacaggttgatgcggataccgttgttgcgcaagattctgctactcgaatggtcgcctcattcttgcatgatacgagagtgattgcactctgcggagagacagctcttaacaacgccaaatcttccatgatcacgatgattcaagtctacgaatactggatctcccacaacttgacgaaagcc

SEQ ID NO: 6 - DNA, 8216 bp, Coccidioides spp.
  misc_feature 6305, 6513, 6604: n = A,T,C or G
atggcgcggccccgtcccatgcccagtcgtctctcccctcgctgccccatcacctgcagtccgacactcaactgacggcccaccttgccagccggtaagagatgatatcgtgtttctcaaagttgtgcgttcaaatttttactgacctatatcatctatctcttagattccatgtctccctccccacagctcgcctttcttcgcatgctctgatatgcctgaatacgtatacatcctcaacaaaaggtcctgatggactcaaggaaggaagcgccatgggggaggcggaggatttggcaaggcgagcctggacaagattggggtcaaggggggaaaatcaagctgttgttttcctgtatgtatccccagttggtgctcgcaacatattctcgacgtcgaatctctctctctccctctttctgacacacttttttctgcgatgctgatctgtcgcagaggtgaatctggctcaggcaaaacaactattcgatctcacctgttatcttcgctcctctcattcacctcaaccccgctatctaccaagttgtcccttgcggcatttgtttttgacacactgacaactacaaaagccgttactacgccaactgcgtcaaaagccggtctctttttcgagctacaatatgatgcatcgtccaccgtaaatccgactttgattggtgggaagcttttagatcaccggctggagcggagcagaatagcgtctgttcctacgggagagcgaagttttcatgttttgtattaccttctggcggggaccagtccagccgaaaagacacatctaggtctttctagttcaggcgatgttatctcgagtggcccaaccaagcgatcttcatcccaagcgtcacacaagcgctggcgatatctcggacacccgacccaattaaaggtcggaatcaacgatgcggaaggattccagcactttaagacggcattgcggaaactggaattcccccgaagcgagatcgcagaaatttgccaaatcctggcttgtatcctgcatatcggacaacttgaattcaccacaggtcaggctacgaggacaggggcggatgaaagtggtggatattcgcacgagggcggtgagaccatcaccgtcgtcaaaaacaaggatgttctaagtattgttgccgccttccttggtctgagtgtcgatgaacttgaaacaagtttgggatataagacgaagacaatccataaagaacgggtaaccgttatgcttgatccccagggcgcccgaacaaacgcagatgggtttgcaaggacactctatgctcttctagttgcctatgtgattgaaaacatcaatcagcggatctgcgctgccgaggacgccgtggctaacaccatttccgttgttgatttccctggttttgcccagatgtctacgacaaattcgaccttggatcaattgctgaacaatgctgctaccgagtccctttatcagtattgtttgcaaaacttctttgagcgtcaggcagatatgttggaaacagaagaggtcaacgtcccagcaaccagctattttgacaactccgatgccgttaagggccttttgaaacacggcaacggtctcctctccattttggacgatcaaaccaagcgcgcccgaaccgacttgcagttcctcgagagtattcgcaaacggttcgagaataaaaacccagctatagctgttggtagctcgaccgcaattctcccaggcagcaactttgctacgcaaaacgctgccgctgtattcacagtgaggcattttgcaggggaagttgattaccccgttaagggcctaattgaagaaaatggtgatctggtatctggcgacctccttaacctcattagctctacgaagagtggatttgttcgggaactgttcggtcaagaagcattgcaaacgatccaccatcccaaggaaaggaatgctatcatgcaagctcaagtcagctcgaaaccattgagaatgccgagcatggcacggcggaaaatgagcagaccctcaaggtttacgtcttttggatctcagtccgaagctgacgacaccgatgactttgctagtacatctaccagtacgaagaaggccggatctaacagtaagaaagggtcagtggctggtgctccaaaacagggcgcagctggccagttcctcgcctctcttgacaatataaccaaatccttatcagcgtcaaatgtgaacccttattttgtcttctgccttaagccaaatgacaggcggattgcaaatcagtttgatagcaaatgcgtccggacccaagtgcagacgttggggattgccgagatcagccaacgtctccgcaatgcggacttctctgtattcttaccatttgctgaattccttgggcttgcagaagcagaatcgatcattgttgggagtgacaaggaaaagtcgcagctcatcgttgatgagaagaggtggccaaccaacgaggcacgcgttggcagcactggtgtcttcctgagtgaaagatgctgggcagatctggccaaagttggcgagcgagttcttccaagttttagtggagaaggtactgatgatggcagagatggccttctccatgttggcggcaaccaatatggagattcaaaagtccgtctcttgccttctggagactccacccctggagcgtttatatacggggatgataagcaaggatacttcggcagccgggaattggatgcacggtcggatgctggcgcatctgcctttaactcaggtgatatgttcaagaatttggaaacccgtgagcaaatggcagagcgaagcaacgagaaagagatggttgaagtggaggacgtcatcgtctcgggaagccgcaaacgctggcttgcgctcgtttatctgttgaccttttatatccctgatttcttgatcaagtgggtcgggagaaagaagagaaaggatgtgcgtgtcgcttggcgtgaaaaattcgctatcaaccttcttatatggttgagctgtggctttgtggtatttttcattatccttttcccgcgattgatctgtccgacgcaacacgttttctccgctgaagaactttcgtctcacgatgggaaaggcaaccataacgcatataccgccattcgaggagaagtatttgacttgggctcttttatacctgcccattaccccctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatgcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgcgggatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagaagtcctcgtgtactgtgtaagcgcccactccacatctccactcgacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggttcgaaagtctttttggctctgtgacctgtttaccaggttgtttcagtatgtaccgcatccgtgccgccgatacaggcaagccactcttcgtcagcaaggaagttgtagatgcctatggagagattcgtgttgatacgcttcacatgaaaaacttgctccatctgggagaggatcgatatctgacaaccctacttctgaagcaccatccaaaatataagaccaaatatattttcaatgcgcatgcatggactgttgctcctgacagctgggccgtcttcttgtcccaacgtcgcagatggatcaattcgactgtccacaacttgatcgagctcattcctctccaacaactgtgcgggttctgctgcttcagtatgaggtttgttgtttttgtggatcttctcagtacaatcattcaacctgtgaccgtcgcctatattgtctacctcattgtactcctggctatgaacaccggcatcattccaatcacagcattcattttgctcggagcgatctacggtctgcaggctattattttcattctccgccgcaaatgggaaatggtaggatggatgataatttatctccttgccatgcctgtgttctcgttgggtctgcctctctatgctttttggcatatggatgacttcacatggggtaatactcgtattgtcactggtgagaaaggccgtaaggtcgttatcacggatgagggaaagtttgatccagcttcaattcctaagaagaagtgggaggagtatcaagctgagctctgggaagcccagacgcatcgtgatgaccagtcggaagtctctggatactcctatcgaacaaagtcgtaccacccagcaatgtcggaatatggatatcccgccacaagaccagcttctcacctggattacaaccgccattcgcggctatctctgcccacttctgaactcctcggtcgtggctcagacctcgaaatgactgacttcgccggtcttccgaccgacgatgcgttgctagccgagatcagagacattctgagaacggctgacttgatgaccgttacgaagaagagcgtcaaacaagaacttgaacggagatttaacatcccgctggacgcaaaacgagcgtacattaattctggtaagttcccttccaaaccagctcgagaaatatggctgttctggcaaaatactaacttacttctgtagcgactgaagcagttctttcaggccaactctaa

SEQ ID NO: 7 - DNA, 30 bp, Artificial Sequence
  Primer
tctacgacaaattcgaccttggatcaattg

SEQ ID NO: 8 - DNA, 30 bp, Artificial Sequence
  Primer
cttcgtaacggtcatcaagtcagccgttct

SEQ ID NO: 9 - DNA, 20 bp, Artificial Sequence
  Primer
acagcgtctccgacctgatg

SEQ ID NO: 10 - DNA, 20 bp, Artificial Sequence
  Primer
cctcgctccagtcaatgacc

SEQ ID NO: 11 - DNA, 20 bp, Artificial Sequence
  Primer
tcctccgcttattgatatgc

SEQ ID NO: 12 - DNA, 22 bp, Artificial Sequence
  Primer
ggaagtaaaagtcgtaacaagg

SEQ ID NO: 13 - DNA, 149 bp, Coccidioides spp.
tggtcgatctcatacatggtctcttggtctctcctttacatttgtgttgggcatgatttttccttttttttggtctctccctttctctctgaaatgaaggtatatatatatccatactccgtacagtagagcaaatttgtttgatacac

SEQ ID NO: 14 - DNA, 22 bp, Artificial Sequence
  Primer
tggtcgatctcatacatggtct

SEQ ID NO: 15 - DNA, 27 bp, Artificial Sequence
  Primer
gtgtatcaaacaaatttgctctactgt

SEQ ID NO: 16 - DNA, 1206 bp, Coccidioides spp.
cagtgctgttcgacttcactcccaccaatatatcgggttctgcgaacgttgttagcggaaacgacaaaaatttcctgtaccatgacttcagacacttcagaaatgatactcgaccaacctggtactttcagcaaatgcgatacctacgggcaaactacaaaaaaggagagattggctacactaagagccatctcaagacgcttgctgagaaggagtctcagtacatcggagtcataaatggagtggtttatgacttcactgactacatgattggaggtcgaattactagagacaaagatggtaaaccgaaaccaggtgtcgacgtggatttcatggattcccgagttgtcgatttgttccagcaaaaatccgggcatgatctgaccgagtacttcaacagacttcccctcagcagcacactgcgttacgatatggaagtctgcctcaacaacctcttcaaagtcggagccctagacacccgaaattcgacgcagtgcctcttctctcaatattttgtgcttgcgtgttcggctctcctggcgagtatcattggttttaagttcttggctgcattgcagtttggaagaaagaatgtcccagaaaaccttgataagtttatcatttgccaggtaccagcgtatacagaagatgaggagtctctccgtcgtgcgattgactcgatggcccggatgaaatacgatgataaacggaaactcctcgtggttatttgcgacggtatgatcattggacagggcaatgaccgccccacgcctcgaatcgtcctcgatatccttggtgtcccagagactgttgaccccgaaccactcagctttgaaagtttaggagaaggcatgaggcagcacaacatgggcaaggtttactccggactgtacgaagtgcaaggtcacattgttccatttttggtagtggttaaagttggcaagccgtctgaggtttctcgacctggtaaccgcggaaaacgtgattctcaaatgcttctgatgcgcttcctcaaccgcgtccattacaacgcaccgatgagcccgcttgaactggaaatgcatcatcaggtccgcaatattattggtgtaaatccgacattctacgagttcatcttacaggttgatgcggataccgttgttgcgcaagattctgctactcgaatggtcgcctcattcttgcatgatacgagagtgattgcactct

SEQ ID NO: 17 - DNA, 540 bp, Coccidioides spp.
acagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcg

SEQ ID NO: 18 - DNA, 745 bp, Coccidioides spp.
gtaaatccgactttgattggtgggaagcttttagatcaccggctggagcggagcagaatagcgtctgttcctacgggagagcgaagttttcatgttttgtattaccttctggcggggaccagtccagccgaaaagacacatctaggtctttctagttcaggcgatgttatctcgagtggcccaaccaagcgatcttcatcccaagcgtcacacaagcgctggcgatatctcggacacccgacccaattaaaggtcggaatcaacgatgcggaaggattccagcactttaagacggcattgcggaaactggaattcccccgaagcgagatcgcagaaatttgccaaatcctggcttgtatcctgcatatcggacaacttgaattcaccacaggtcaggctacgaggacaggggcggatgaaagtggtggatattcgcacgagggcggtgagaccatcaccgtcgtcaaaaacaaggatgttctaagtattgttgccgccttccttggtctgagtgtcgatgaacttgaaacaagtttgggatataagacgaagacaatccataaagaacgggtaaccgttatgcttgatccccagggcgcccgaacaaacgcagatgggtttgcaaggacactctatgctcttctagttgcctatgtgattgaaaacatcaatcagcggatctgcgctgccgaggacgccgtggctaacaccatttccgttgttgatttccctggttttgcccaga

SEQ ID NO: 19 - DNA, 25 bp, Artificial Sequence
  Primer
gtaaatccgactttgattggtggga

SEQ ID NO: 20 - DNA, 27 bp, Artificial Sequence
  Primer
tctgggcaaaaccagggaaatcaacaa

SEQ ID NO: 21 - DNA, 517 bp, Coccidioides spp.
aagttctcactcctcagcgctatcgcagcggctgtcttcgtccctttcacatccgccactccacttgctagcacggccgacctcagctacgacactcactacgatgacccatccctgcccctgagtggcgtcacctgttctgacggggacaatggcatgataacaaagggctacaacaccgccggcgagataccaaactaccctcacgtcggaggagcttttacggtcgaaacgtggaacagccccaactgtggaaagtgctacaaagtgacatacaatgctaaaacgatttttttgactgcgatcgaccacagcaactccggatttaatatcgcgaagaagtcgatggacgtattgacgaacggacgggcagaggaattgggcaggatcaaggtgacctacgaagaggtcgcctcgtcgttgtgcgggttgaaataaaggcgtattgggcgatgtgccgcaatgctgagtgcgatgatttgatatttgtttggttgaaggggaggaaccttaatgt

SEQ ID NO: 22 - DNA, 25 bp, Artificial Sequence
  Primer
aagttctcactcctcagcgctatcg

SEQ ID NO: 23 - DNA, 25 bp, Artificial Sequence
  Primer
acattaaggttcctccccttcaacc

SEQ ID NO: 24 - DNA, 20 bp, Artificial Sequence
  Primer
tcctccgcttattgatatgc

SEQ ID NO: 25 - DNA, 22 bp, Artificial Sequence
  Primer
ggaagtaaaagtcgtaacaagg

SEQ ID NO: 26 - DNA, 3055 bp, Coccidioides spp.
ctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaacaatggcgagacacagatgacttccgaaagagtatgacttgtcttaacaaccaatttcacgtcggcatattagatttcagagaaacaccccgatgtggggtcaacaactacattcttctggcgttcaccattattctttgttccgttattctcgtcaagttccttgcggctctccagctgggaaccaaacggcgaccctcacctcaggataaattcgtcatctgccttgtgccggcgtatacagaaggtgaggatcagcttcgcaagggacttgattcgcttacagcgctgcaatatgacaacaaacgaaaactcatctgtgtcgtctgcgatggtatgattgttggtggaggtaacgacaggcctacgccgaagattgtgccggacattttgggcgttgacccaaagattgatccgccagcccttccgttcaagtcagttggcgatgggagcgaacagcttaactatggcaaggtctactccggcctttacgaatatgaaggaaatgttgttccatatattggcgtcgtcaaagtcggaaagcaatccgaacagggcaagtcaaagccaggaaaccgtggaaagcgtgattcgcaggttatgcttcttaatttcttgaaccgcgtgcaccatcgctcgctcatgtctccactggaactggaaatgttccatcaaatcaacaacgtaattggtgtcgacccagaactttacgaatacgtgtttatggtcgacgccgacactagtgttagagaagactcgctgaatcgtcttgttgcaagctgcgcgaacgacgccaaaattgctggtatttgtggtgagacgagcttgcaaaatgaagagcgttcttggtggaccatgattcgggtctatgaatattacatctctcatcatttggcaaaatctttcgaatcgctctttggtagtgtgacctgtcttccagggtggtaagttgtcttttccccttttgatgtacgtccttgagtaccacgcttaattcttcgcacagcttctgcatgtatcgactccggacagcagacaagggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtc

SEQ ID NO: 27 - DNA, 25 bp, Artificial Sequence
  Primer
ctcgtgaatcgccaatcacaaatgc

SEQ ID NO: 28 - DNA, 25 bp, Artificial Sequence
  Primer
gacgtgtggcgggagttgcgagcct

SEQ ID NO: 29 - DNA, 6071 bp, Artificial Sequence
  Recombinant Plasmid
gggcgaattgggcccgacgtcgcatgctcccggccgccatggcggccgcgggaattcgattctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaacaatggcgagacacagatgacttccgaaagagtatgacttgtcttaacaaccaatttcacgtcggcatattagatttcagagaaacaccccgatgtggggtcaacaactacattcttctggcgttcaccattattctttgttccgttattctcgtcaagttccttgcggctctccagctgggaaccaaacggcgaccctcacctcaggataaattcgtcatctgccttgtgccggcgtatacagaaggtgaggatcagcttcgcaagggacttgattcgcttacagcgctgcaatatgacaacaaacgaaaactcatctgtgtcgtctgcgatggtatgattgttggtggaggtaacgacaggcctacgccgaagattgtgccggacattttgggcgttgacccaaagattgatccgccagcccttccgttcaagtcagttggcgatgggagcgaacagcttaactatggcaaggtctactccggcctttacgaatatgaaggaaatgttgttccatatattggcgtcgtcaaagtcggaaagcaatccgaacagggcaagtcaaagccaggaaaccgtggaaagcgtgattcgcaggttatgcttcttaatttcttgaaccgcgtgcaccatcgctcgctcatgtctccactggaactggaaatgttccatcaaatcaacaacgtaattggtgtcgacccagaactttacgaatacgtgtttatggtcgacgccgacactagtgttagagaagactcgctgaatcgtcttgttgcaagctgcgcgaacgacgccaaaattgctggtatttgtggtgagacgagcttgcaaaatgaagagcgttcttggtggaccatgattcgggtctatgaatattacatctctcatcatttggcaaaatctttcgaatcgctctttggtagtgtgacctgtcttccagggtggtaagttgtcttttccccttttgatgtacgtccttgagtaccacgcttaattcttcgcacagcttctgcatgtatcgactccggacagcagacaagggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtcatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactata

SEQ ID NO: 30 - DNA, 5034 bp, Artificial Sequence
  PCR Product of Recombinant Plasmid
agggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtcatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgattctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaac

SEQ ID NO: 31 - DNA, 25 bp, Artificial Sequence
  Primer
agggccgaccactgattatatccga

SEQ ID NO: 32 - DNA, 25 bp, Artificial Sequence
  Primer
gttccgtaatgtcggcaccagcatt

SEQ ID NO: 33 - DNA, 1037 bp, Artificial Sequence
  PCR Product of Recombinant Plasmid
aatggcgagacacagatgacttccgaaagagtatgacttgtcttaacaaccaatttcacgtcggcatattagatttcagagaaacaccccgatgtggggtcaacaactacattcttctggcgttcaccattattctttgttccgttattctcgtcaagttccttgcggctctccagctgggaaccaaacggcgaccctcacctcaggataaattcgtcatctgccttgtgccggcgtatacagaaggtgaggatcagcttcgcaagggacttgattcgcttacagcgctgcaatatgacaacaaacgaaaactcatctgtgtcgtctgcgatggtatgattgttggtggaggtaacgacaggcctacgccgaagattgtgccggacattttgggcgttgacccaaagattgatccgccagcccttccgttcaagtcagttggcgatgggagcgaacagcttaactatggcaaggtctactccggcctttacgaatatgaaggaaatgttgttccatatattggcgtcgtcaaagtcggaaagcaatccgaacagggcaagtcaaagccaggaaaccgtggaaagcgtgattcgcaggttatgcttcttaatttcttgaaccgcgtgcaccatcgctcgctcatgtctccactggaactggaaatgttccatcaaatcaacaacgtaattggtgtcgacccagaactttacgaatacgtgtttatggtcgacgccgacactagtgttagagaagactcgctgaatcgtcttgttgcaagctgcgcgaacgacgccaaaattgctggtatttgtggtgagacgagcttgcaaaatgaagagcgttcttggtggaccatgattcgggtctatgaatattacatctctcatcatttggcaaaatctttcgaatcgctctttggtagtgtgacctgtcttccagggtggtaagttgtcttttccccttttgatgtacgtccttgagtaccacgcttaattcttcgcacagcttctgcatgtatcgactccggacagcagaca

SEQ ID NO: 34 - DNA, 3465 bp, Artificial Sequence
  PCR Product of Recombinant Vector
gaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagagcttggcac

SEQ ID NO: 35 - DNA, 25 bp, Artificial Sequence
  Primer
gaattcccttgtatctctacacaca

SEQ ID NO: 36 - DNA, 25 bp, Artificial Sequence
  Primer
gtgccaagctctagaaagaaggatt

SEQ ID NO: 37 - DNA, 8499 bp, Artificial Sequence
  Recombinant Plasmid
agggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtcatcactagtgaattcgcggccgcctgcaggtcgaccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggcggccgcgggaattcgattctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaacgaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagagcttggcac

SEQ ID NO: 38 - DNA, 5483 bp, Artificial Sequence
  PCR Product of Recombinant Plasmid
ctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaacgaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagagcttggcacagggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtc

SEQ ID NO: 39 - DNA, 25 bp, Artificial Sequence
  Primer
ctcgtgaatcgccaatcacaaatgc

SEQ ID NO: 40 - DNA, 25 bp, Artificial Sequence
  Primer
gacgtgtggcgggagttgcgagcct

SEQ ID NO: 41 - DNA, 668 bp, Coccidioides spp.
gtcaacaactacattcttctggcgttcaccattattctttgttccgttattctcgtcaagttccttgcggctctccagctgggaaccaaacggcgaccctcacctcaggataaattcgtcatctgccttgtgccggcgtatacagaaggtgaggatcagcttcgcaagggacttgattcgcttacagcgctgcaatatgacaacaaacgaaaactcatctgtgtcgtctgcgatggtatgattgttggtggaggtaacgacaggcctacgccgaagattgtgccggacattttgggcgttgacccaaagattgatccgccagcccttccgttcaagtcagttggcgatgggagcgaacagcttaactatggcaaggtctactccggcctttacgaatatgaaggaaatgttgttccatatattggcgtcgtcaaagtcggaaagcaatccgaacagggcaagtcaaagccaggaaaccgtggaaagcgtgattcgcaggttatgcttcttaatttcttgaaccgcgtgcaccatcgctcgctcatgtctccactggaactggaaatgttccatcaaatcaacaacgtaattggtgtcgacccagaactttacgaatacgtgtttatggtcgacgccgacactagtgttagagaagactcgctgaatcg

SEQ ID NO: 42 - DNA, 25 bp, Artificial Sequence
  Primer
gtcaacaactacattcttctggcgt

SEQ ID NO: 43 - DNA, 25 bp, Artificial Sequence
  Primer
cgattcagcgagtcttctctaacac

SEQ ID NO: 44 - DNA, 599 bp, Coccidioides spp.
ctacgagaacgggaacgagacagaatacaaggagtgcttgagaaagttggagttgaattagaatccggggctttcctgaaaaaggtgctatatcctgacgaaggaggagatctgaactttggcagaaggggacgcttcgacctcaatgctactcttggtagcggcagggtctggtatcacctctcaatgcacccggttgatgaccacccggctgcaatcgcatctctaccctcggtcagctcggcgtggtcagcagggacagtttcgcgtcaactacgtgcttggagacttgctgaatgggccaatcgccgcaacaaacaccttgacttcaccgcagactttgacgttgacgaattcgttagtcgatatgctcttctaggctgccacgaaggcaaagatggcgtggaaaattggatcattgaacgaggctggagtaacggtgatgttgtagtcgcaagggagagaatctggatgcgtgaaggggcttggtgggaggcagaatcgatgctcgacttgaaaattccaacggcacctgtaaatcccttcgccaccccggcggctactactcccttcgatggaacctacggagctccgtcacc

SEQ ID NO: 45 - DNA, 25 bp, Artificial Sequence
  Primer
ctacgagaacgggaacgagacagaa

SEQ ID NO: 46 - DNA, 25 bp, Artificial Sequence
  Primer
ggtgacggagctccgtaggttccat

SEQ ID NO: 47 - DNA, 508 bp, Coccidioides spp.
atcaagataatcctgccaatgccagccgtctcagtggagttggaatgcttactcccgacggccatccaggagtctctcagcgacagagtatgcgtagtcccctgtcacggccagccagcaccatgatcgatttcagaaatccaccaaatcagggaccggacgagactgctattacagctgctatccggtcttgtcttgcggaggtggatttggacagtgtgaccaagaagcaaggttcgtacatttgatattgaccacactgaccgttaaatatgctaacgatcatgtagtccgagcgcttgttgagcaaaggttgcagacaacattgacgggcgacaaaaaagccttccttgatcgccaaatcgaccacgaattggcaaatatgtaaaatcctttgtgctaatcaagcaccacttgcccactctcggaacacacacctgtttcatactaaccttgcaacttgattttgataatcctggtcggggaccggactaatctatgcatgatt

SEQ ID NO: 48 - DNA, 25 bp, Artificial Sequence
  Primer
atcaagataatcctgccaatgccag

SEQ ID NO: 49 - DNA, 25 bp, Artificial Sequence
  Primer
aatcatgcatagattagtccggtcc

SEQ ID NO: 50 - DNA, 500 bp, Coccidioides spp.
agactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatc

SEQ ID NO: 51 - DNA, 25 bp, Artificial Sequence
  Primer
agactctagcttggagcatagaggg

SEQ ID NO: 52 - DNA, 25 bp, Artificial Sequence
  Primer
gatggtcgttgtaggggctgtatta

SEQ ID NO: 53 - DNA, 7926 bp, Coccidioides posadasii
  misc_feature 6066, 6274, 6365: n = A,T,C or G
atggcgaaccgcatgtcagtatattctacctcatcgtccggtccagggccccgccccgccggacagcaagcatcacaggtttcgaccaccacgctgctcaacgcccttcatacctgctatctctccggacaaccttatcagcttgactctgcaacgagcatagtcgtgaacaccgctttaacagctactacgccagggcacaatggcgagctgggcggcactatagatcttaacgtagcgcggagagcgtgggagcatgcccgacggagagcagaggatggatgtattgtattatggtttgtcttgacatattcctatactttcgccccaaacagctaacaactctgtatttagctcgtcgcatcaaaatacaccctctctccttccagcctttttggcatccttgccattacccacaccggagatagcctatacagccctagccgcaattcgacccttcgtttctcaagtcaccccgttcgaccccgcccgcttgctctacccttcctttgccgtgacttacacgttcgccctgacaggaagagtcactgatgtaaacttagcactgtcgacatctggtgtcaacatcaccgatgggctcgcgaggattccatccgaacctggccatcgtgcatttgatgtcttttactacctcctctcctcggcatcgacaccggcagaaagagagttcctcggcctgaaaagaccagaggcgtattccttccttaacaggtctcaaacttacgatccaccatcctacctccctaccaccgacgatggcgcctccgcagaggatttccgagccaatctcaaagctatcggcatcaaaggagcggcccatcgcagcttgctctcagtcctggccggtttgcttaaattgggcaacaccgcggggttcctcgtcgaagaagacgaattggaagacctctgcgaggacatcgggggccttctcaatgtcgatcctatcgttcttctcaaaaactgctcgacagaagaccgagagtcgctcgtcttgggtttatacgagaccctccttgactgggtcatttcgaaagcgaacgaagccattcgcctggagttacatgcctcgcatgataatgggtcgagcgatggcagacccagtgctcgaacacctctaagtgaagaagagagtggagatacggttagcatcactgttgtggatatcccggacgaggcactaggaaaagctgtcgctctgcgaggcgtatttggtgattctctcggcataaatgctgaaatgaaagaggatggaatcaacatggttaacccaggtcatgctatcgttaaggaaatggaaaatgccgtagctgaggtggaacctgagctgggtatcatgggtggcaatgccggtcgcctacgagaacgggaacgagacagaatacaaggagtgcttgagaaagttggagttgaattagaatccggggctttcctgaaaaaggtgctatatcctgacgaaggaggagatctgaactttggcagaaggggacgcttcgacctcaatgctactcttggtagcggcagggtctggtatcacctctcaatgcacccggttgatgaccacccggctgcaatcgcatctctaccctcggtcagctcggcgtggtcagcagggacagtttcgcgtcaactacgtgcttggagacttgctgaatgggccaatcgccgcaacaaacaccttgacttcaccgcagactttgacgttgacgaattcgttagtcgatatgctcttctaggctgccacgaaggcaaagatggcgtggaaaattggatcattgaacgaggctggagtaacggtgatgttgtagtcgcaagggagagaatctggatgcgtgaaggggcttggtgggaggcagaatcgatgctcgacttgaaaattccaacggcacctgtaaatcccttcgccaccccggcggctactactcccttcgatggaacctacggagctccgtcaccagcgacggcaagtggattcttccccccagataacatgagcactttaggcagccgagagaatctcgtgaatcgccaatcacaaatgccaagagcacctagcgcccttggaggaactcgttccattgcgcctactaccgtatctcagcctgttcaggtttcgtctggagactacggccttggactcaagggtgacgaccgacaacatgaaaacgtttattacgatcaagaccttggtaaatggactgctttcgatcccgagtttggagacccgaaaaagatcgaggagaagaaaataccaaccagccgccgcgtttggtctgcttttgtatgggctgtcacgttttggattccatcatttgttcttcgtttcgtgggtcggatgaagcgcccagacgttcgaatggcctggcgtgaaaaagtgactctcatcctccttattttccttctcaacgcttcaattgtattctacatcgtcttcctcggtgacctactctgtccgaacaaagacaaagtctggacggataaagaggttagctatcatcagggcgaaaatgatttctatgtcagcgtccacgggaaggtctacgatataagcaagttttggaagattactcacaccactaaccgatatcccacaacaaaagatcagatgatacattttgctgggaagaatatggacgcgtatttcccgctgcccctttcttacgcttgtcctggatttgatattggcaagcaaattgaattgcaacacaatgatacgagtgctgttgaattcccgcaggctgtccataaggctggtcccgaattccaacccgatcctactcttgcactgagaagaattgattggtatgcaaggacctttttgcccagaatgaaggagtattacaaaggagatctggtgcattcgaggaagtccttaccccaggaagccgaagagcgttctaggcaatgggcatccatcatggtaagagtttacgacttgaccgactacttttataccgtgggagtgcagaacaacctgaaacaatacgacttccttcctcgagccgtaaccgacctgtttaaaaataatgctggtgccgacattacggaacgaattcccttgtatctctacacacaggctcaaatcaataagaagaacggttcgtctttttcgtttatatcttgcatcgtcccaaagctattggcgggatattctgtttgcagttggctgacttgaagtaatctctgcagatctttcgacactgaaatacgtcgagcctgctccgcttggaagcggcgaggagcctcgtcctgtcacaactaccaacatggagtacgataagggccagttccgccagctcattaagagccagttcatgggcgttggcatgatggccgtcatgcatctgtacttcaagtacaccaacgctcttctgatccagtcgatcatccgctgaaggcgctttcgaatctggttaagatccacgtcttcgggaagccagcgactggtgacctccagcgtccctttaaggctgccaacagctttctcagccagggccagcccaagaccgacaaggcctccctccagaacgccgagaagaactggaggggtggtgtcaaggaggagtaagctccttattgaagtcggaggacggagcggtgtcaagaggatattcttcgactctgtattatagataagatgatgaggaattggaggtagcatagcttcatttggatttgctttccaggctgagactctagcttggagcatagagggtcctttggctttcaatattctcaagtatctcgagtttgaacttattccctgtgaaccttttattcaccaatgagcattggaatgaacatgaatctgaggactgcaatcgccatgaggttttcgaaatacatccggatgtcgaaggcttggggcacctgcgttggttgaatttagaacgtggcactattgatcatccgatagctctgcaaagggcgttgcacaatgcaagtcaaacgttgctagcagttccaggtggaatgttatgatgagcattgtattaaatcaggagatatagcatgatctctagttagctcaccacaaaagtcagacggcgtaaccaaaagtcacacaacacaagctgtaaggatttcggcacggctacggaagacggagaagccaccttcagtggactcgagtaccatttaattctatttgtgtttgatcgagacctaatacagcccctacaacgaccatcaaagtcgtatagctaccagtgaggaagtggactcaaatcgacttcagcaacatctcctggataaactttaagcctaaactatacagaataagataggtggagagcttataccgagctcccaaatctgtccagatcatggttgaccggtgcctggatcttcctatagaatcatccttattcgttgacctagctgattctggagtgacccagagggtcatgacttgagcctaaaatccgccgcctccaccatttgtagaaaaatgtgacgaactcgtgagctctgtacagtgaccggtgactctttctggcatgcggagagacggacggacgcagagagaagggctgagtaataagccactggccagacagctctggcggctctgaggtgcagtggatgattattaatccgggaccggccgcccctccgccccgaagtggaaaggctggtgtgcccctcgttgaccaagaatctattgcatcatcggagaatatggagcttcatcgaatcaccggcagtaagcgaaggagaatgtgaagccaggggtgtatagccgtcggcgaaatagcatgccattaacctaggtacagaagtccaattgcttccgatctggtaaaagattcacgagatagtaccttctccgaagtaggtagagcgagtacccggcgcgtaagctccctaattggcccatccggcatctgtagggcgtccaaatatcgtgcctctcctgctttgcccggtgtatgaaaccggaaaggccgctcaggagctggccagcggcgcagaccgggaacacaagctggcagtcgacccatccggtgctctgcactcgacctgctgaggtccctcagtccctggtaggcagctttgccccgtctgtccgcccggtgtgtcggcggggttgacaaggtcgttgcgtcagtccaacatttgttgccatattttcctgctctccccaccagctgctcttttcttttctctttcttttcccatcttcagtatattcatcttcccatccaagaacctttatttcccctaagtaagtactttgctacatccatactccatccttcccatcccttattcctttgaacctttcagttcgagctttcccacttcatcgcagcttgactaacagctaccccgcttgagcagacatcaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggagcttgagatccacttaacgttactgaaatcatcaaacagcttgacgaatctggatataagatcgttggtgtcgatgtcagctccggagttgagacaaatggtgttcaggatctcgataagatacgttcatttgtccaagcagcaaagagtgccttctagtgatttaatagctccatgtcaacaagaataaaacgcgttttcgggtttacctcttccagatacagctcatctgcaatgcattaatgcattgactgcaacctagtaacgccttncaggctccggcgaagagaagaatagcttagcagagctattttcattttcgggagacgagatcaagcagatcaacggtcgtcaagagacctacgagactgaggaatccgctcttggctccacgcgactatatatttgtctctaattgtactttgacatgctcctcttctttactctgatagcttgactatgaaaattccgtcaccagcncctgggttcgcaaagataattgcatgtttcttccttgaactctcaagcctacaggacacacattcatcgtaggtataaacctcgaaatcanttcctactaagatggtatacaatagtaaccatgcatggttgcctagtgaatgctccgtaacacccaatacgccggccgaaacttttttacaactctcctatgagtcgtttacccagaatgcacaggtacacttgtttagaggtaatccttctttctagagcttggcacagggccgaccactgattatatccgacaaggttatcgccgaatatgctgacggtgatgttgatacactgcacaagaagaaccttttatccttgggagaagatcgatacctcacgacattgatgaccaagcatttcccaagtatgtcgtacaagttcatccccgacgcatacgccagcacagctgcaccggaaacctggagcgttttgctttcacaaagacgtcgttggatcaattccacgatccacaatctggcagaattgatgttcctcaaggacctgtgcggcttctgttgctttagcatgcgttttattgtgttcatcgacctgtttggtacgatcatcctcccggcgacaacagtatacttgggatatctcatctatcgtgtggcaagtggcacgggtcaattccctgtgatctcaatcgcgatcctcgcagccgtctacggtttacaggccatcattttcattatcaaacgtcaatggcaacacattggatggatgatcatctacatcatggcctacccaatttacagttttatcctccccatttattcgttctggaaccaagacaatttctcatggggtaatacccgtattgtccttggcgaaaagggtgataagcgagtcgttgcggtggatgacgaaggatttgaccctcgaagcattcctcttcaacgctgggacgactatgctctcatgaacaatcttcctggtcgccgaggtaattccatgggccaggaaaagcctcatcagcaatattacgatgacggagggctcgagatggacgatatgcattcggtttactcgtcagtgaagccagcatctacgattctaaccggattccctaatcagggcatgcaccacacaccctatgtcctcccgcactctacaaatcctttggctgttccaggcaacagaaactcccatatgacgcagttcactcaatacactgataatccccaggctcgcaactcccgccacacgtcgattggaaatctatgtaaatatcaagataatcctgccaatgccagccgtctcagtggagttggaatgcttactcccgacggccatccaggagtctctcagcgacagagtatgcgtagtcccctgtcacggccagccagcaccatgatcgatttcagaaatccaccaaatcagggaccggacgagactgctattacagctgctatccggtcttgtcttgcggaggtggatttggacagtgtgaccaagaagcaaggttcgtacatttgatattgaccacactgaccgttaaatatgctaacgatcatgtagtccgagcgcttgttgagcaaaggttgcagacaacattgacgggcgacaaaaaagccttccttgatcgccaaatcgaccacgaattggcaaatatgtaa

(19) United States
(12) Patent Application Publication
(10) Pub. No.: US 2005/0260233 A1
(43) Pub. Date: Nov. 24, 2005

(54) Attenuated vaccine useful for immunizations against Coccidioides spp. infections

(75) Inventors: Garry T. Cole, Toledo, OH (US); Charles N. Okeke, Toledo, OH (US); Chiung-Yu Hung, Toledo, OH (US)

Correspondence Address: MORRISON & FOERSTER LLP, 425 MARKET STREET, SAN FRANCISCO, CA, 94105-2482, US

(73) Assignee: MEDICAL COLLEGE OF OHIO, Toledo, OH (US)

(21) Appl. No.: 11/102,217

(22) Filed: Apr. 7, 2005

Related U.S. Application Data
(60) Provisional application No. 60/560,512, filed on Apr. 7, 2004.

(51) Int. Cl. C12Q 1/68; C12N 1/18; A61K 39/00

(52) U.S. Cl. 424/274.1; 435/254.1

(57) ABSTRACT

The present invention provides compositions of attenuated fungal mutants and polynucleotide sequences used to transform fungal strains by gene deletions or gene replacements, which are useful for generating an immunological response in human and animals and in therapeutic applications of infections due to pathogenic Coccidioides spp. fungi, such as C. immitis or C. posadasii.

### RELATED APPLICATION

This application claims benefit of priority under 35 U.S.C. 119(B) of Provisional application 60/560,512 filed Apr. 7, 2004, the contents of which are incorporated herein by reference.

### STATEMENT REGARDING FEDERALLY FUNDED PROJECT

The United States Government owns rights in the present invention pursuant to Public Service Grants “Immunoreactive Macromolecules of Coccidioides Cell Types” (AI19149) and “Isolation and Expression of Coccidioides T-cell Antigens” (AI37232) from the National Institutes of Allergy and Infectious Diseases, National Institutes of Health.

### FIELD OF THE INVENTION

The present invention relates generally to the fields of pathogenic fungi and immunology. In particular, the invention provides compositions of Coccidioides spp. strains attenuated by the selective targeting and replacement of genes encoding proteins necessary for the morphogenic conversion and growth of parasitic phase propagules. More particularly, the present invention provides compositions of Coccidioides spp. recombinant strains, which are useful for generating an immunological response in an individual and in vaccines and therapeutic applications of infections due to pathogenic Coccidioides spp. fungi, such as C. posadasii or C. immitis.

### BACKGROUND OF THE INVENTION

Coccidioidomycosis, otherwise known as the San Joaquin Valley Fever, is a fungal respiratory disease of humans and wild and domestic animals, which is endemic to southwestern United States, northern Mexico, and numerous semiarid areas of Central and South America (Pappagianis, D. Epidemiology of Coccidioidomycosis. Current Topics in Medical Mycology. 1988. 2:199-23). Infection occurs by inhalation of airborne spores (arthroconidia) produced by the saprobic phase of Coccidioides spp., which grows in alkaline desert soil, followed by morphogenic conversion of the fungus to the virulent, parasitic phase in the host mammal.

Coccidioides immitis was the first described species, and is now becoming known as the Californian species. The C. posadasii species was recently defined, and was previously recognized as the non-Californian population of C. immitis (Fisher, M. C., Koenig, G. L., White, T. J., Taylor, J. W. Molecular and phenotypic description of Coccidioides posadasii sp. nov., previously recognized as the non-California population of Coccidioides immitis. Mycologia 2002. 94(1):73-84, 2002). The differences in the two species are slight.

It is estimated that 100,000 new cases of this disease occur annually within the rapidly growing population of people who live in regions of the United States between southwest Texas and southern California, where the disease is endemic (Galgiani, J. N. Coccidioidomycosis: A regional disease of national importance; rethinking our approaches to its control. Annals of Internal Medicine. 1999. 130:293-300). Although the majority of immunocompetent individuals are able to resolve their Coccidioides spp. infection spontaneously, the level of morbidity associated even with the primary form of this respiratory mycosis warrants consideration of a vaccine against the disease. Immunocompromised patients, including those infected with human immunodeficiency virus, are at high risk to contract disseminated coccidioidomycosis (Ampel, N. M., C. L. Dols, and J. N. Galgiani. Results of a prospective study in a coccidioidal endemic area. American Journal of Medicine. 1993. 94:235-240). It is also apparent from results of several clinical studies that African-Americans and Asians are genetically predisposed to development of the potentially fatal, disseminated form of the respiratory disease (Galgiani, J. N. 1993. Coccidioidomycosis. Western Journal of Medicine 159:153-171).

The rationale for commitment of research efforts to develop a Coccidioides spp. vaccine is based on clinical evidence that individuals who recover from the respiratory coccidioidomycosis disease retain effective long-term cellular immunity against future infections by the pathogen (Smith, C. E. 1940. American Journal of Public Health 30:600-611). In addition, early preclinical studies demonstrated that a formalin-killed whole-cell (spherule) vaccine prevented deaths in mice after infection with even very large numbers of coccidioidal spores (Levine et al.1961. Journal of Immunology 87:218-227). However, when a similar vaccine preparation was evaluated in a human trial, there was substantial local inflammation, pain, and induration at the injection site, rendering the vaccine unacceptable (Pappagianis et al. Evaluation of the protective efficacy of the killed Coccidioides immitis spherule vaccine in humans. American Review of Respiratory Diseases. 1993.148:656-660). Further, there was no difference in the number of cases of coccidioidomycosis or the severity of the disease in the formalin-killed spherule vaccinated group compared to the placebo group. Therefore, the original human vaccine trial was not successful.

Other attempts to identify a suitable vaccine have focused on the creation of attenuated, live strains of C. immitis for the induction of an immune response. In two such attempts, investigators induced auxotrophic mutations in strains of C. immitis via X-ray irradiation (Foley, J. M, Berman, R. J., and Smith, C. E. X-ray irradiation of Coccidioides immitis arthrospores: survival curves and avirulent mutants isolated. Journal of Bacteriology. 1960. 79:480) or UV-irradiation and chemical mutagenesis (Walch, H. A. and Walch. R. K. Studies with induced mutants of Coccidioides immitis. In L. Ajello (ed.) Proceedings of the Second Symposium on Coccidioidomycosis. University of Arizona Press. 1960. p 339), and then utilized the attenuated strains as vaccines prior to challenging the animals with wild-type C. immitis. However, these and subsequent reports utilizing these strains (Pappagianis, D., Levine, H. B., Smith, C. E., Berman, R. J. and Kobayahsi, G. S. Immunization of mice with viable Coccidioides immitis. Journal of Immunology. 1961. 86:28; Walch, H. A. and Walch. R. K. Immunization of mice with induced mutants of Coccidioides immitis. I. Characterization of mutants and preliminary studies of their use as viable vaccines. Sabouraudia. 1971. 9:173) demonstrated that although varying degrees of immunization were attained with these strains, the attenuated strains nevertheless were capable of converting to the parasitic phase and resulted in localized or disseminated lesions in the experimental animals. In one instance, the attenuated strain regained its virulence through the loss of the auxotrophic state, causing disease in vaccinated animals. Given the evidence of localized or disseminated disease, the investigators found the attenuated strains to be inappropriate as vaccines.

Therefore, there is a long felt need for more effective and safer vaccines to prevent, treat, or ameliorate infection of Coccidioides spp. and disease states associated with the infection.

### SUMMARY OF THE INVENTION

Accordingly, it is an object herein to provide the methods for creating attenuated strains of Coccidioides spp. that have an immunostimulatory activity. As used herein, the term “attenuated” is used in the broadest sense to mean to render a fungus strain less virulent or less capable of causing coccidioidomycosis in a mammal. It is understood by those skilled in the art that such attenuated strain may be capable of growth under artificial in vitro conditions, or may be capable of limited growth when introduced into a mammal, but are of insufficient pathogenic potential to cause disease. Examples of an attenuated fungus can be found in U.S. Pat. No. 6,248,322, which by reference is incorporated herein in its entirety. Such immunostimulatory attenuated strains will be useful in the prevention and treatment of infections due to Coccidioides spp. In one embodiment, the attenuated Coccidioides spp. fungus is Coccidioides posadasii. In another embodiment, the attenuated Coccidioides spp. fungus is Coccidioides immitis.

In order to meet these needs, compositions and methods for the production of attenuated strains of Coccidioides spp. have been devised that render the strains incapable of transforming from the saprophytic, mycelial form to the parasitic, spherule-endospore phase of the fungus. Thus, such strains are replication competent, meaning that they have the ability to reproduce as mycelia in the saprophytic phase in vitro, but are incapable of growth in the virulent parasitic phase. Such strains are otherwise intact, but their inability to undergo morphogenic conversion results in the strain's inability to cause disease.

Surprisingly, we have found that such strains of attenuated fungus are capable of inducing a potent immune response. Accordingly, another aspect of the invention provides a method for causing a mammal to resist infection by Coccidioides spp. by administration to the mammal the attenuated fungus by single or multiple injections in a manner sufficient to elicit a protective immune response against the wild type fungus. Preferably, the administration of the attenuated fungus is by subcutaneous or intramuscular injection. In one embodiment, the recombinant fungus provides protection against Coccidioides posadasii and or Coccidioides immitis infections in a mammal, such as a human. In another embodiment, the recombinant fungus provides protection against Coccidioides spp. infection in domestic animals, including but not limited to dogs, cats, horses, and cattle.

Further, we have found that the selective introduction of genetic alterations in certain genes, leading to the loss of functional proteins encoded by those genes, results in the loss of morphogenic conversion potential in Coccidioides spp. Such genes include but are not limited to those known to have differential expression in the different growth phases of Coccidioides spp.; for example genes encoding the proteins CHS5, CHS7, HSP70, HSP104, HSP82, HSP90, HSP26, beta-glucosidase 3, beta-glucosidase 5, and parasitic phase-specific protein PSP-1. Additionally, such genes include but are not limited to the Coccidioides spp. orthologs of genes known to control cell wall development or morphogenesis in other fungi, for example genes encoding the following proteins: Psu1, a cell wall synthesis protein reported to be essential for cell wall synthesis in fission yeast (GenBank AB009980, Biochem Biophys Res Commun. 262(2): 368-741, 1999); verprolin (Vrp), involved in cytoskeletal organization and cellular growth in Saccharomyces cerevisiae, (GenBank Ref|xp_{—}324261.1, Mol Microbiol. 10(3):585-96, 1993); DigA, a protein in Aspergillus nidulans required for nuclear migration, mitochondrial morphology and polarized growth. (GenBank Ref|np_{—}588498.1, Mol Genet Genomics.266(4):672-685, Epub 2001); FluG, a protein reportedly essential for asexual development of Aspergillus nidulans (GenBank AAC37414.1, Genetics, Vol.158, 1027-1036, 2001); Ras2, a RAS related GTP-binding protein that controls morphogenesis, pheromone response, and pathogenicity in the plant fungal pathogen Ustilago maydis (GenBank AY149917, Eukaryotic Cell 1 (6): 954-966, 2002); HymA, a protein essential for the development of conidiophore in Aspergillus nidulans (GenBank AJ001157, Mol Gen Genet. 260(6):510-21, 1999).

In one example, strains of recombinant, replication competent Coccidioides posadasii fungus have been modified to render them incapable of expressing a functional CHS5 protein. The CHS5 protein is one of several homologs of chitin synthase found in Coccidioides spp. that regulate chitin synthesis; a key structural component of the cell wall of the fungus. As used herein, homolog means a second gene within the same species derived from a common ancestral gene that has evolved a new, though similar function.

Compositions and methods are provided for the production of a CHS5 null mutant comprising a transformed strain of Coccidioides spp. created by a targeted replacement of polynucleotides from genomic CHS5 with the recombinant sequence comprising but not limited to nucleotides 37 to 6673 of SEQ ID NO:4. In one embodiment, the targeted replacement results in a transformed strain of Coccidioides spp. with the CHS5 null mutant incorporating the recombinant sequence of SEQ ID NO:6. In another embodiment, the attenuated fungus is created by the a deletion from the genomic CHS5 of a polynucleotide sequence comprised of, but not limited to, SEQ ID NO:5.

The present invention also provides polynucleotides encoding the CHS5 null mutant, produced by recombinant technology from the CHS5 gene and gene fragments derived from Coccidioides posadasii, including but not limited to the polynucleotide sequence of SEQ ID NO:4.

In one embodiment, the attenuated fungus is incapable of producing a functional CHS5-encoded protein. In another embodiment, the attenuated fungus is incapable of transcribing the polynucleotide sequence of SEQ ID NO:6 and or translating the resulting transcript into a polypeptide.

The invention also provides compositions in which a second mutation is introduced to further attenuate the fungus.

The present invention further provides the use of the recombinant fungus in combination with one or more other Coccidioides spp. polypeptides to elicit an immune response sufficient to provide an effective immunization against Coccidioides spp. infection. In one embodiment the recombinant fungus and polypeptides are provided as a composition containing a mixture of said fungus and polypeptides. In another embodiment the composition is provided as separate compositions to be administered concurrently or consecutively; the latter consistent with the well-known practice of “prime-boost” for eliciting an immune response.

The present invention further provides methods and compositions of recombinant fungal strains identical or substantially identical to the recombinant C. posadasii strains containing the polynucleotide sequence of SEQ ID NO:6 useful in pharmaceutical compositions.

The present invention also provides vaccine formulations and methods of preparing the formulations containing the attenuated fungus. The present invention further provides vaccine formulations containing adjuvants and pharmaceutical excipients and carriers.

The present invention provides the attenuated Coccidioides spp. fungus vaccine formulations and methods for eliciting an effective immune response in a mammal, including humans and domestic animals, for the prevention of Coccidioides spp. infections.

The present invention further provides kits containing the attenuated Coccidioides spp. fungus, to facilitate the use of the fungus for eliciting an effective immune response in a mammal.

The above and other aspects of the invention will become readily apparent to those of skill in the art from the following detailed description and figures, wherein only the preferred embodiments of the invention are shown and described, simply by way of illustration of the best mode of carrying out the invention. As is readily recognized, the invention is capable of modifications within the skill of the relevant art without departing from the spirit and scope of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Nucleotide (SEQ ID NO:1) and derived amino acid sequences (SEQ ID NO:2) of the native C. posadasii CHS5 gene [amino acids are displayed in a single-letter abbreviation after alignment for maximal identity by the program CLUSTAL W (PAM 250 matrix)]. The open reading frame (ORF) of CHS5 deduced from the genomic sequence is interrupted by three introns, from nucleotides 900-971, 1161-1261 and 6520-6587, respectively. The translated amino acid sequence of Chs5p shows a protein of 1857 residues.

FIG. 2: The nucleotide sequence (SEQ ID NO:4) of the 6734-bp Spe I/Apa 1, linear fragment of pΔCHS5 comprising 36 bp of pCR2.1-TOPO (nt 1-36), 1759-bp CHS5 fragment (nt 37-1795), 3822-bp pAN7-1 fragment (nt 1796-5617), 1056-bp CHS5 fragment (nt 5618-6673), and 61 bp pCR2.1-TOPO (nt 6674-6734), used in the transformation of C. posadasii protoplast.

FIG. 3: The nucleotide sequence (SEQ ID NO:6) of the 8216-bp recombinant CHS5 gene in the transformed mutant of C. posadasii, wherein bp 1-1406 is the 5′ CHS5 fragment outside the crossover region; bp 1407-3165 is the left-flank crossover region; bp 3166-6987 is a pAN7-1 fragment; bp 6988-8043 is the right-flank crossover region; and bp 8044-8216, 3′ CHS5 fragment outside the crossover region.

### BRIEF DESCRIPTION OF THE SEQUENCE LISTING

SEQ ID NO:1 depicts the determined nucleotide sequence of CHS5 open reading frame (ORF);

SEQ ID NO:2 depicts the derived amino acid sequence of the native Chs5 polypeptide encoded by the nucleotide sequence of SEQ ID NO:1;

SEQ ID NO:3 depicts the nucleotide sequence a 4236-bp fragment of CHS5 that was amplified by PCR using the primers DIS-F [SEQ ID:6] and DIS-R [SEQ ID:7], and used for the construction of pΔCHS5;

SEQ ID NO:4 depicts the nucleotide sequence of a 6734-bp Spe I/Apa I-linearized fragment of pΔCHS5 comprising 36 bp of pCR2.1-TOPO (nt 1-36), 1759-bp CHS5 fragment (nt 37-1795), 3822-bp pAN7-1 fragment (nt 1796-5617), 1056-bp CHS5 fragment (nt 5618-6673), and 61 bp pCR2.1-TOPO (nt 6674-6734), used in the transformation of C. posadasii protoplasts;

SEQ ID NO:5 depicts the nucleotide sequence of the 1420-bp fragment of CHS5 deleted from the Δchs5 genome;

SEQ ID NO:6 depicts the nucleotide sequence of the 8216 bp recombinant CHS5 gene;

SEQ ID NO:7 depicts the forward primer used for the PCR amplification of SEQ.ID-3;

SEQ ID NO:8 depicts the reverse primer used for the PCR amplification of SEQ.ID-3;

SEQ ID NO:9 depicts the nucleotide sequence of the PN-1 forward primer derived from the HPH gene of pAN7-1, and used for PCR confirmation of integration of pΔCHS5 in Δchs5 genome;

SEQ ID NO:10 depicts the nucleotide sequence of the PN-2 reverse primer derived from the HPH gene of pAN7-1, and used for PCR confirmation of integration of pΔCHS5 in Δchs5 genome;

SEQ ID NO:11 depicts the nucleotide sequence of the GW5-F2 forward primer derived from the deleted fragment of CHS5, and used for PCR confirmation of deletion of CHS5 fragment in Δchs5 genome;

SEQ ID NO:12 depicts the nucleotide sequence of the GW6-2R reverse primer derived from the deleted fragment of CHS5, and used for PCR confirmation of deletion of CHS5 fragment in Δchs5 genome;

SEQ ID NO:13 depicts the nucleotide sequence of the PA digoxigenin-labeled probe, which was amplified by PCR from the 3′ end of CHS5 outside the crossover region, and used in southern analysis to confirm the homologous integration of pΔCHS5 fragment at the CHS5 locus;

SEQ ID NO:14 depicts the nucleotide sequence of the forward primer used for the PCR amplification of the probe of SEQ.ID-13;

SEQ ID NO:15 depicts the nucleotide sequence of the reverse primer used for the PCR amplification of the probe of SEQ.ID-13;

SEQ ID NO:16 depicts the nucleotide sequence of the Prb X digoxigenin-labeled probe, which was amplified by PCR using the primers of SEQ.ID:10 and SEQ.ID:11 derived from the targeted deletion locus of CHS5, and used in southern analysis to confirm the deletion of the 1420-bp CHS5 polynucleotide fragment of SEQ.ID:5 in the Δchs5 genome;

SEQ ID NO:17 depicts the nucleotide sequence of the Prb Y digoxigenin-labeled probe, which was amplified by PCR using the primers of SEQ.ID:9 and SEQ.ID:10, and used for Southern blot confirmation of integration of pΔCHS5 in Δchs5 genome;

SEQ ID NO:18 depicts the nucleotide sequence of the PZ digoxigenin-labeled probe, which was amplified by PCR from the 5′ end of CHS5 outside the crossover region, and used in southern analysis to confirm the homologous integration of pΔCHS5 fragment at the CHS5 locus;

SEQ ID NO:19 depicts the nucleotide sequence of the forward primer used for the PCR amplification of SEQ.ID:18;

SEQ ID NO:20 depicts the nucleotide sequence of the reverse primer used for the PCR amplification of SEQ.ID:18;

SEQ ID NO:21 depicts the nucleotide sequence of the 517 bp CSA fragment, which was amplified by PCR and used to confirm the identity of C. posadasii transformants;

SEQ ID NO:22 depicts the nucleotide sequence of the forward primer used in the PCR amplification of the sequence of SEQ ID NO:21;

SEQ ID NO:23 depicts the nucleotide sequence of the reverse primer used in the PCR amplification of the sequence of SEQ ID NO:21;

SEQ ID NO:24 depicts the nucleotide sequence of the universal fungal primer derived from 18S rDNA, and used in the PCR amplification of the ITS1 and ITS2 regions of the rDNA for the confirmation of the identity of C. posadasii transformants;

SEQ ID NO:25 depicts the nucleotide sequence of the universal fungal primer derived from 28S rDNA, and used in the PCR amplification of the ITS1 and ITS2 regions of the rDNA for the confirmation of the identity of C. posadasii transformants.

SEQ ID NO:26 depicts the nucleotide sequence of the 3055-bp fragment of CHS7 amplified by PCR from C. posadasii genomic DNA using the primers of SEQ ID NO:27 and SEQ ID NO:28;

SEQ ID NO:27 depicts the forward primer used for the PCR amplification of SEQ.ID:26;

SEQ ID NO:28 depicts the reverse primer used for the PCR amplification of SEQ.ID NO:26;

SEQ ID NO:29 depicts the nucleotide sequence of the plasmid pGEM-CHS7 comprising a 3055-bp fragment of CHS7 (SEQ ID.NO.26) cloned into the 3016-bp plasmid vector, pGEM-T EASY;

SEQ ID NO:30 depicts the nucleotide sequence of a PCR product of pGEM-CHS7, using the primers of SEQ ID NO:31 and SEQ ID NO:32, yielding a 5034-bp fragment, pGEM-CHS7-1037 from which 1037-bp fragment (SEQ ID NO:33) of CHS7 was deleted;

SEQ ID NO:31 depicts the forward primer used for the PCR amplification of SEQ.ID:30;

SEQ ID NO:32 depicts the reverse primer used for the PCR amplification of SEQ.ID NO:30;

SEQ ID NO:33 depicts the nucleotide sequence of a 1037 bp-fragment of CHS7 deleted from PCR product of SEQ ID NO:30;

SEQ ID NO:34 depicts the nucleotide sequence of a PCR product of the transformation vector, pAN8-1, using the primers of SEQ ID NO:35 and SEQ ID NO:36;

SEQ ID NO:35 depicts the forward primer used for the PCR amplification of SEQ.ID:34;

SEQ ID NO:36 depicts the reverse primer used for the PCR amplification of SEQ.ID NO:34;

SEQ ID NO:37 depicts the nucleotide sequence of the CHS7 gene deletion plasmid vector, pGEM-AN8-CHS7, comprising fragments of the fungal transformation vector pAN8-1 (bp 5035-8499) ligated to fragments of CHS7 (bp 1-985 and bp 4002-5034) and pGEM (bp 986-4001), for the transformation of Escherichia coli strain TAM 1.;

SEQ ID NO:38 depicts the nucleotide sequence of CHS7-pAN8-CHS7, a linearized 5483-bp fragment of SEQ ID NO:37 produced by PCR using primers SEQ ID NO:39 and SEQ ID NO:40, for the transformation of protoplasts of C. posadasii;

SEQ ID NO:39 depicts the forward primer used for the PCR amplification of SEQ.ID:38;

SEQ ID NO:40 depicts the reverse primer used for the PCR amplification of SEQ ID NO:38.

SEQ ID NO:41 depicts the nucleotide sequence of a 668-bp probe (Probe 1) used for Southern analysis determination of a CHS7 deletion;

SEQ ID NO:42 depicts the nucleotide sequence of the forward primer used for the PCR amplification of probe 1 (SEQ ID NO:41);

SEQ ID NO:43 depicts the nucleotide sequence of the reverse primer used for the PCR amplification of probe 1 (SEQ ID NO:41);

SEQ ID NO:44 depicts the nucleotide sequence of a 659 bp probe (Probe 2) located at the 5′ UTR outside of the cross over region of CHS7;

SEQ ID NO:45 depicts the nucleotide sequence of the forward primer used for the PCR amplification of Probe 2 (SEQ ID NO:44);

SEQ ID NO:46 depicts the nucleotide sequence of the reverse primer used for the PCR amplification of Probe 2 (SEQ ID NO:44);

SEQ ID NO:47 depicts the nucleotide sequence of a 508 bp probe (Probe 3) located at the 3′UTR outside of the crossover region of CHS7;

SEQ ID NO:48 depicts the nucleotide sequence of the forward primer used for the PCR amplification of Probe 3 (SEQ ID NO:47);

SEQ ID NO:49 depicts the nucleotide sequence of the reverse primer used for the PCR amplification of Probe 2 (SEQ ID NO:47);

SEQ ID NO:50 depicts the nucleotide sequence of a 500 bp probe (Probe 4) used in Southern analysis confirmation of pAN8-1 integration in the mutant genome;

SEQ ID NO:51 depicts the nucleotide sequence of the forward primer used for the PCR amplification of Probe 4 (SEQ ID NO:50);

SEQ ID NO:52 depicts the nucleotide sequence of the reverse primer used for the PCR amplification of Probe 2 (SEQ ID NO:50);

SEQ ID NO:53 depicts the nucleotide sequence of the recombinant CHS7 in CHS7-null mutant of C. posadasii.

### DETAILED DESCRIPTION

Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausubel et al (eds.), John Wiley & Sons, New York (2001) and the various references cited therein.

I. The Attenuated Fungi of the Invention.

Techniques to interfere with the regulatory mechanisms that control morphogenic conversion to the parasitic, virulent form of the Coccidioides spp. fungus are described herein. Methods have been devised that render the strains of the fungus incapable of transforming from the saprophytic, mycelial form to the parasitic, spherule-endospore phase of the fungus. Thus, such strains are replication competent, retaining the ability to reproduce as mycelia in the saprophytic phase in vitro, but are incapable of growth in the virulent parasitic phase. Because the mutant strains are incapable of sustained growth and reproduction in the parasitic form of the Coccidioides spp. fungus when introduced into a mammal, the strains are incapable of causing disease, rendering them attenuated. Since the attenuated strains retain properties necessary for their immunogenicity and safety, such strains would be useful for a preventative or therapeutic vaccine for coccidioidomycosis.

The general approach was to identify suitable sites for genetic alteration of the chromosome of the Coccidioides spp. fungus in order to create knockouts of genes, selecting those that controlled the morphogenic conversion from the saprophytic to the parasitic phase, rendering them attenuated, while retaining properties of the strain necessary for its immunogenicity and safety.

More specifically, using the methods and approaches described in Example 4 and those known in the art, for example, Sambrook et al., Molecular Cloning: A Laboratory Manual, Third Edition, Cold Spring Harbor Laboratory Press, New York (2001), Current Protocols in Molecular Biology, Ausubel et al (eds.), John Wiley & Sons, New York (2001) and the various references cited therein, attenuated mutant strains would be created by transformation of wild-type strain Coccidioides spp. with gene deletion plasmid vectors designed to delete, by double crossover events, polynucleotide sequences of genes essential for regulation of morphogenic conversion in Coccidioides spp. A group of genes for such knock-out strains includes but is not limited to those known to have differential expression in the different growth phases of Coccidioides spp.; for example genes encoding the proteins CHS5, CHS7, HSP70, HSP104, HSP82, HSP90, HSP26, beta-glucosidase 3, beta-glucosidase 5, parasitic phase-specific protein PSP-1. The genetic sequences encoding said proteins would be obtained as disclosed herein from a public database available at The Institute for Genomic Research (TIGR) web site at tigr.org using computational analyses of the partial genome database by application of the basic local alignment search tool (BLAST) (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Research 25:3389-3402). Another group of genes for such knock-out strains includes but is not limited to the Coccidioides spp. orthologs of genes known to control cell wall development or morphogenesis in other fungi, for example genes encoding the following proteins: Psu1, a cell wall synthesis protein reported to be essential for cell wall synthesis in fission yeast (GenBank AB009980, Biochem Biophys Res Commun. 262(2): 368-741, 1999); verprolin (Vrp), involved in cytoskeletal organization and cellular growth in Saccharomyces cerevisiae, (GenBank Ref|xp_{—}324261.1, Mol Microbiol. 10(3):585-96, 1993); DigA, a protein in Aspergillus nidulans required for nuclear migration, mitochondrial morphology and polarized growth. (GenBank Ref|np_{—}588498.1, Mol Genet Genomics.266(4):672-685, Epub 2001); FluG, a protein reportedly essential for asexual development of Aspergillus nidulans (GenBank AAC37414.1, Genetics, Vol. 158,1027-1036, 2001); Ras2, a RAS related GTP-binding protein that controls morphogenesis, pheromone response, and pathogenicity in the plant fungal pathogen Ustilago maydis (GenBank AY149917, Eukaryotic Cell 1 (6): 954-966, 2002); HymA, a protein essential for the development of conidiophore in Aspergillus nidulans (GenBank AJ001157, Mol Gen Genet. 260(6):510-21, 1999). As used herein, ortholog means genes in different species that evolved from a common ancestral gene by speciation that retain the same or essentially the same function in the course of evolution. The known sequences from the non-Coccidioides fungi would be used to conduct BLAST searches of the Coccidioides posadasii sequence data available at the TIGR database in order to obtain the corresponding Coccidioides ortholog gene sequences.

The Coccidioides gene sequences would then be used to create sequence alignments using the translated nucleotide sequences of the contigs or complete gene sequences and the non-redundant protein database available from the National Center for Biotechnology Information (Wheeler, D. L., C. Chappey, A. E. Lash, D. D. Leipe, T. L. Madden, G. D. Schuler, T. A. Tatusova, and B. A. Rapp. 2000. Database resources of the National Center for Biotechnology Information. Nucleic Acids Research 28:10-14), BLASTX matches would be selected with Expect (E) values of <10⁴ as previously described (Kirkland, T. N., and G. T. Cole. 2002. Gene-finding in Coccidioides immitis: searching for immunogenic proteins, p. 247-254. In K. J. Shaw (ed.), Pathogen genomics: impact on human health. Humana Press, Totowa, N.J.). Once appropriate sequences are derived, using the methods described herein appropriate transforming plasmids would be constructed and the Coccidioides spp. wild-type strain would be transformed to selectively replace and or delete polynucleotide sequences of individual genes that encode the targeted polypeptides using plasmids designed to result in a double crossover event.

Using the methods described more fully in the Examples, the identity and homology of the mutant transformants would be confirmed by PCR, sequence analysis, and Southern blot analysis by the methods described herein and would be subsequently screened to confirm loss of morphogenic potential to the parasitic phase and such strains would be evaluated for confirmation of lack of virulence in the mouse model previously described. Strains demonstrating lack of virulence would be considered attenuated and would be subsequently screened to confirm their immunogenicity in the vaccination mouse model previously described in Example 4. If the vaccination experiment showed increased survival in mice vaccinated with the attenuated strain and challenged with the wild-type strain and a reduction in the recovery of viable fungus from the organs of necropsied mice, this would confirm the strain as an attenuated vaccine useful for prevention of coccidioidomycosis.

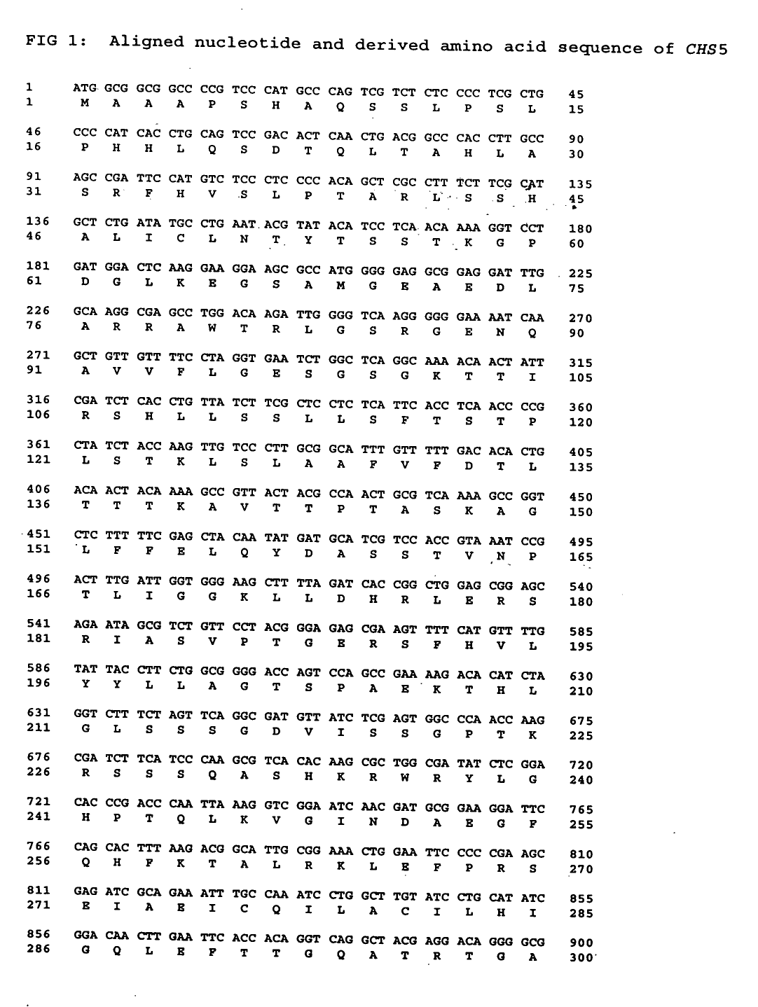

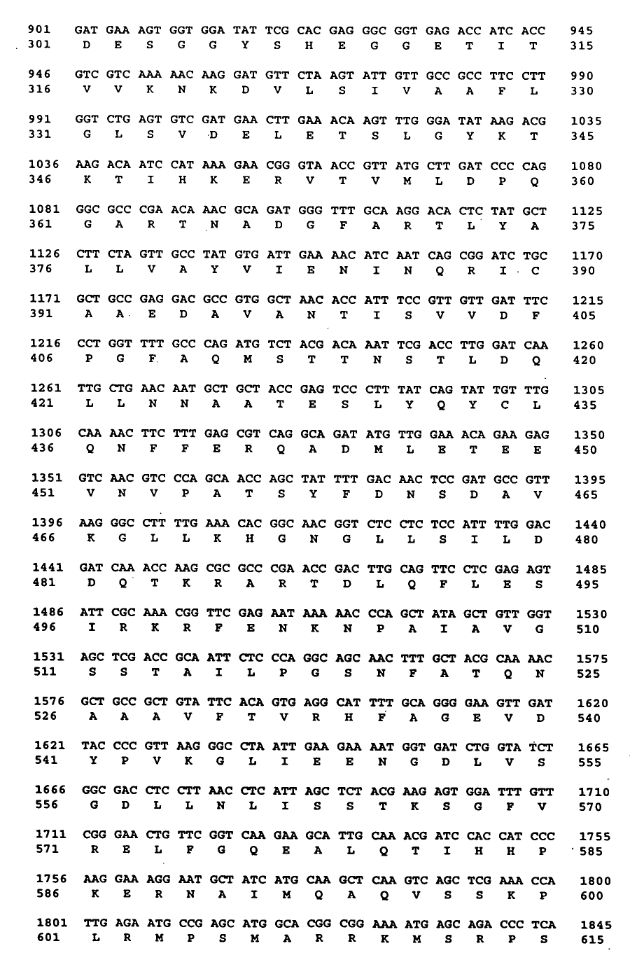

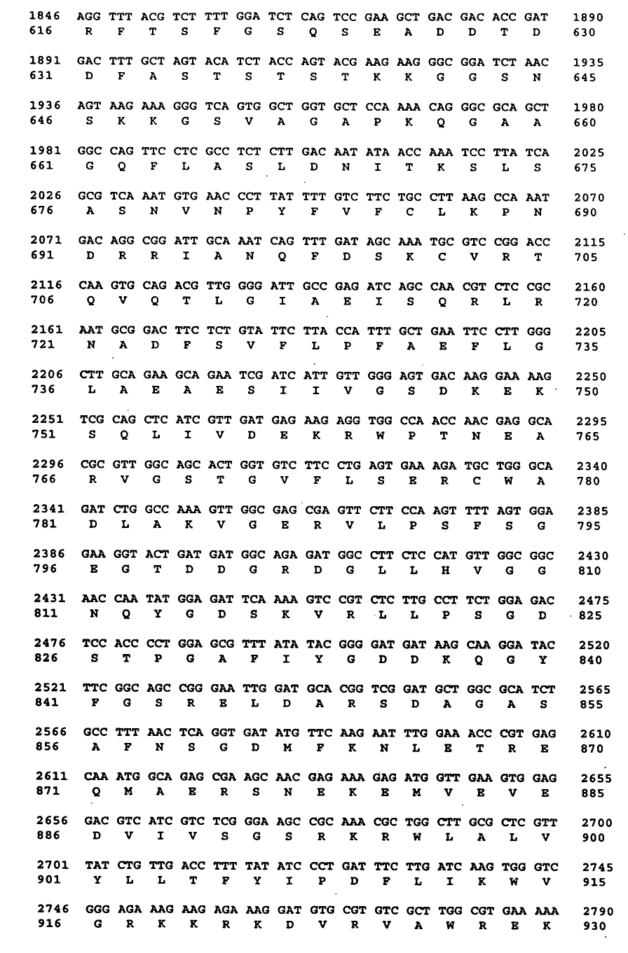

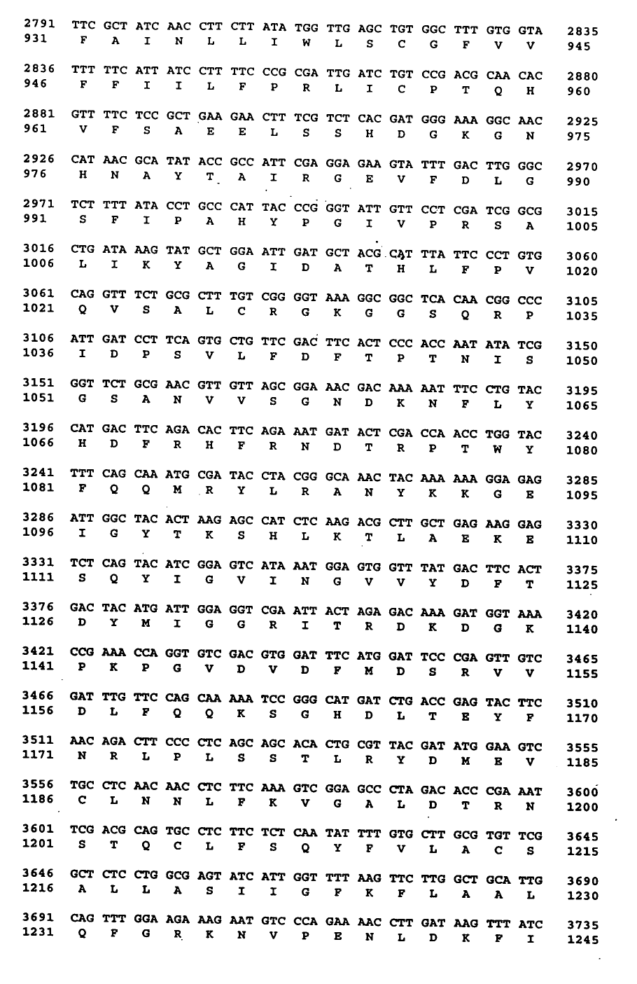

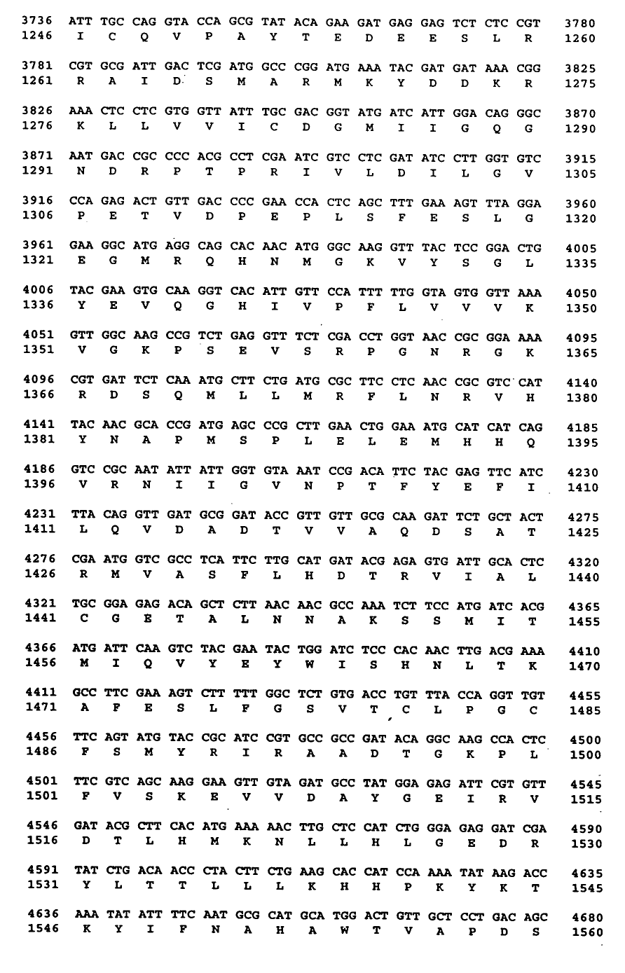

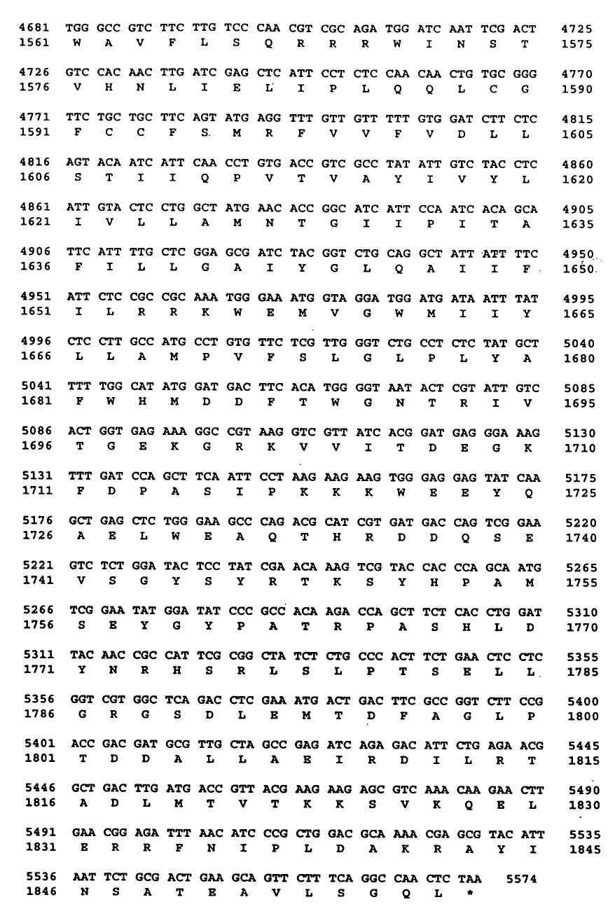

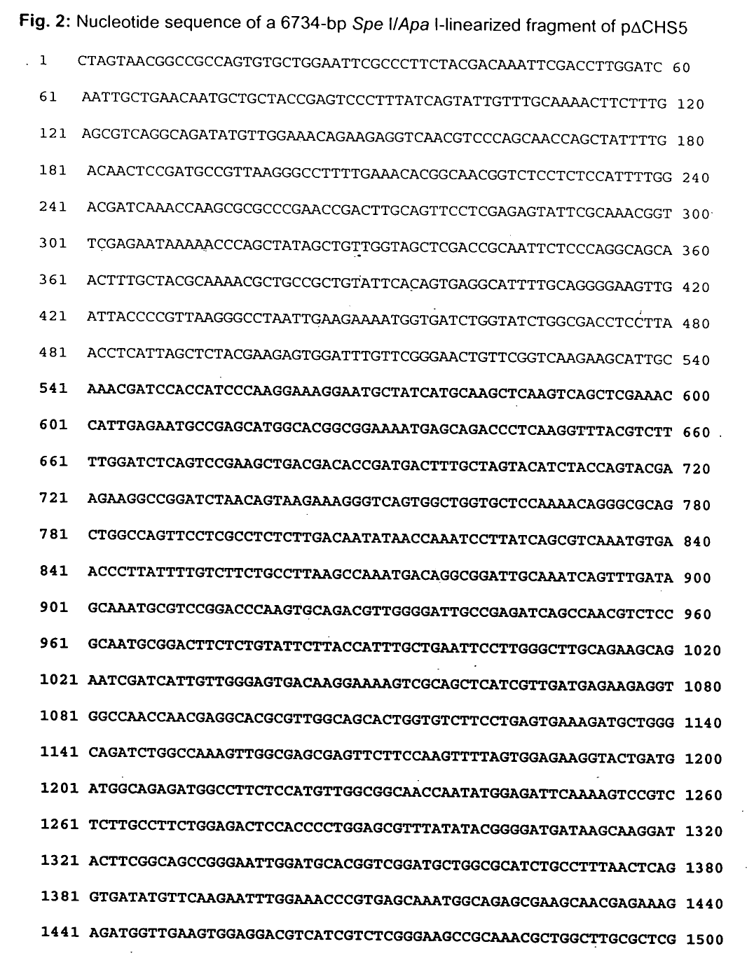

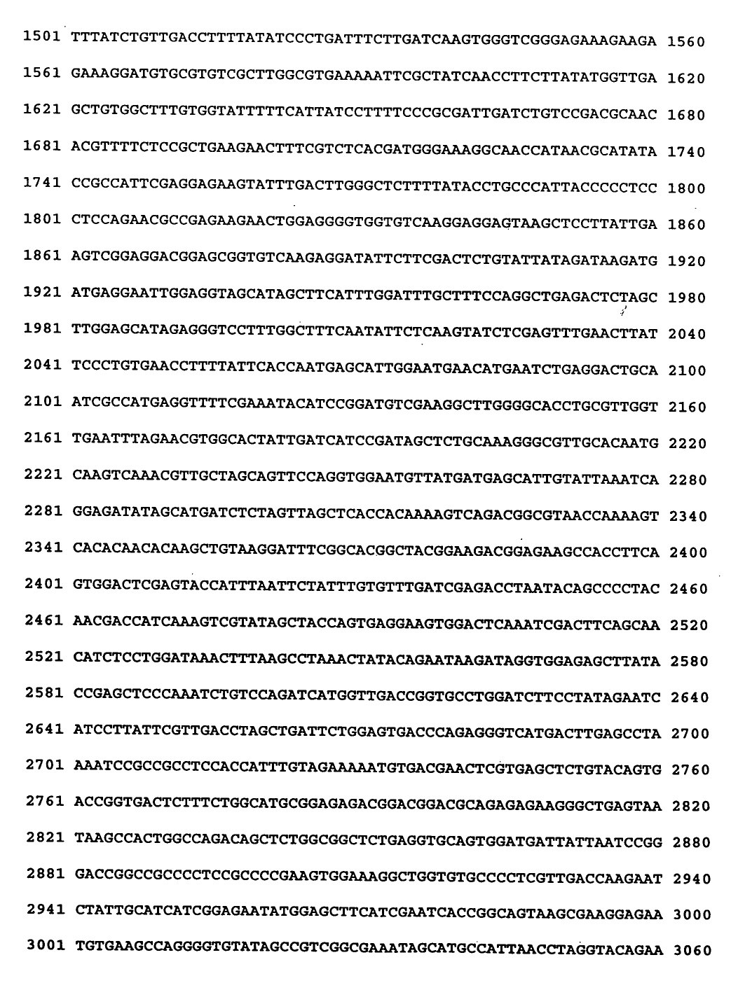

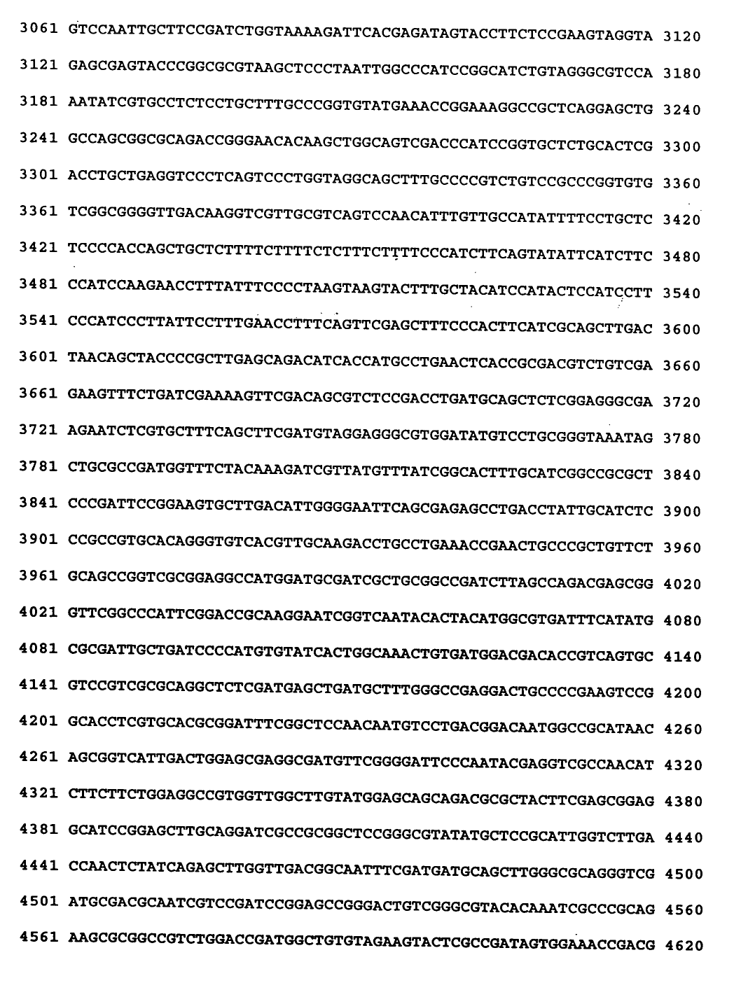

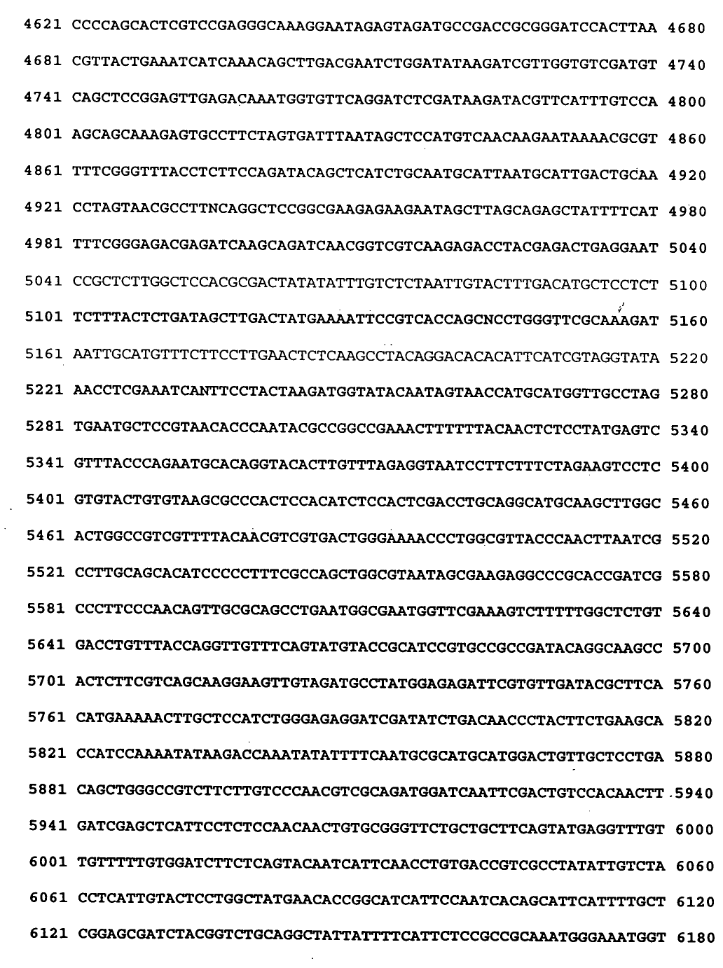

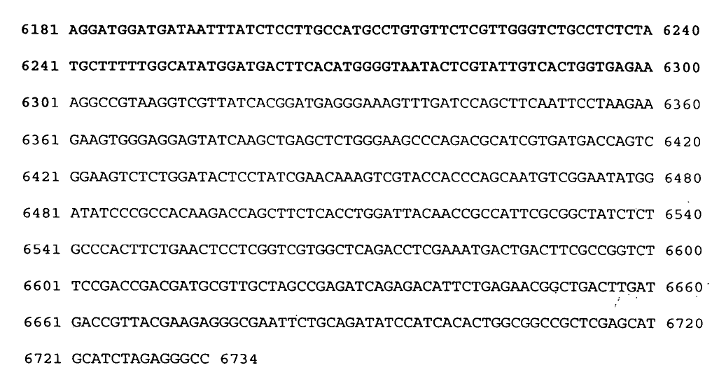

A specific embodiment of the molecular strategy is a CHS5 knockout of the Coccidioides spp. fungus. Functional analyses of CHS gene expression in several fungal pathogens and disruption phenotypes have shown differences in the contribution of individual isozymes to fungal growth and viability (Liu H, Wang Z, Zheng L, Hauser M, Kauffman S, Becker J M, Szaniszlo P J. 2001. Relevance of chitin and chitin synthases to virulence in Wangiella (Exophiala) dermatitidis, a model of melanized pathogen of humans, p. 463-472. In: R. A. A. Muzzarelli (ed.), Chitin Enzymology 2001. Atec Edizioni, Italy). Of the seven known chitin synthases found in Coccidioides spp. (Okeke C N, Cole G T. 2003. Morphological defects and loss of virulence of Coccidioides posadasii caused by disruption of CHS5, encoding a chitin synthase with a myosin motor-like domain. 103^{rd} General Meeting of the American Society for Microbiology, Abstract F-023.; Cole G T, Hung C Y. 2001. The parasitic cell wall of Coccidioides immitis. Medical Mycology 39 Supplement 1:31-40), the class 5 chitin synthase is a unique protein containing an N-terminal myosin motor-like domain and is possibly involved in cytoskeleton-mediated translocation of the enzyme to the sites of cell wall or septal synthesis (Munro C A, Gow N A R. 2001. Chitin synthesis in human pathogenic fungi. Medical Mycology 39 Suppl. 1: 41-53). The knockout of the CHS5 gene of the Coccidioides spp. fungus was performed using targeted gene replacement in the wild-type gene sequence shown in FIG. 1, resulting in homologous integration of a hygromycin resistance cassette by double crossover recombination at the flanking CHS5-homologous fragments, and the consequent deletion of an internal CHS5 fragment. The mutated CHS5 locus of the CHS5-null mutant contained the transformation plasmid construct shown in FIG. 2, but lacks a significant fragment of CHS5, rendering the resulting translated polypeptide nonfunctional. The rest of the fungus DNA was left intact. By this approach, the fungus maintained an ability to grow, making it replication competent in vitro, but could not undergo morphogenic conversion into the virulent, parasitic form capable of causing disease in a mammal. Importantly, the attenuated strain retained immunogenic potential, thereby making it suitable for use as a preventative or therapeutic vaccine.

II. The DNA Sequences of the Invention.

The methods utilized for selective replacement and or deletion of polynucleotide sequences in strains of Coccidioides spp. fungus, leading to the corresponding loss amino acids necessary for functional proteins critical to morphogenic potential of the fungus and, hence, virulence, are disclosed herein. Polynucleotide coding sequences for amino acid residues are known in the art and are disclosed for example in Molecular Cloning: A Laboratory Manual, Third Edition, Sambrook, Fritsch, and Maniatis, Cold Spring Harbor Laboratory Press, 2001.

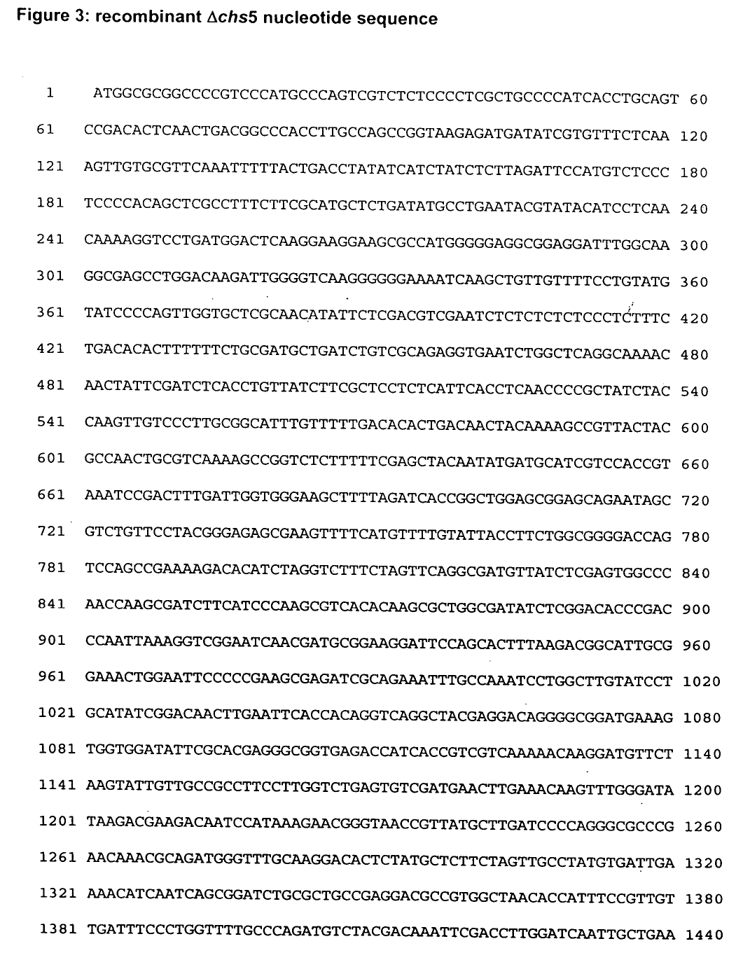

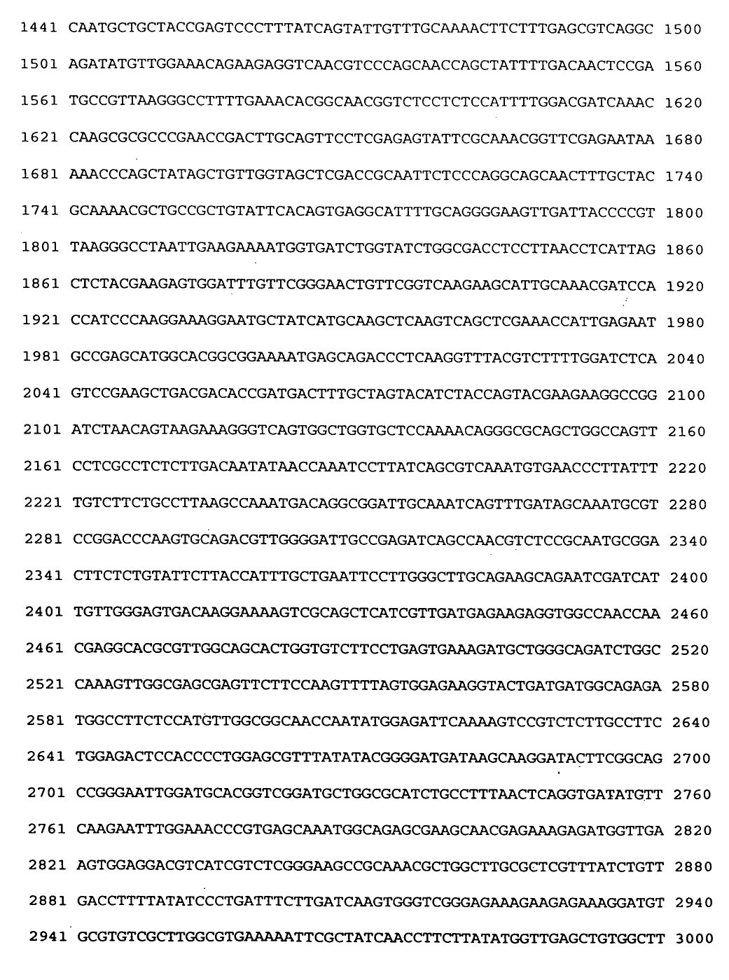

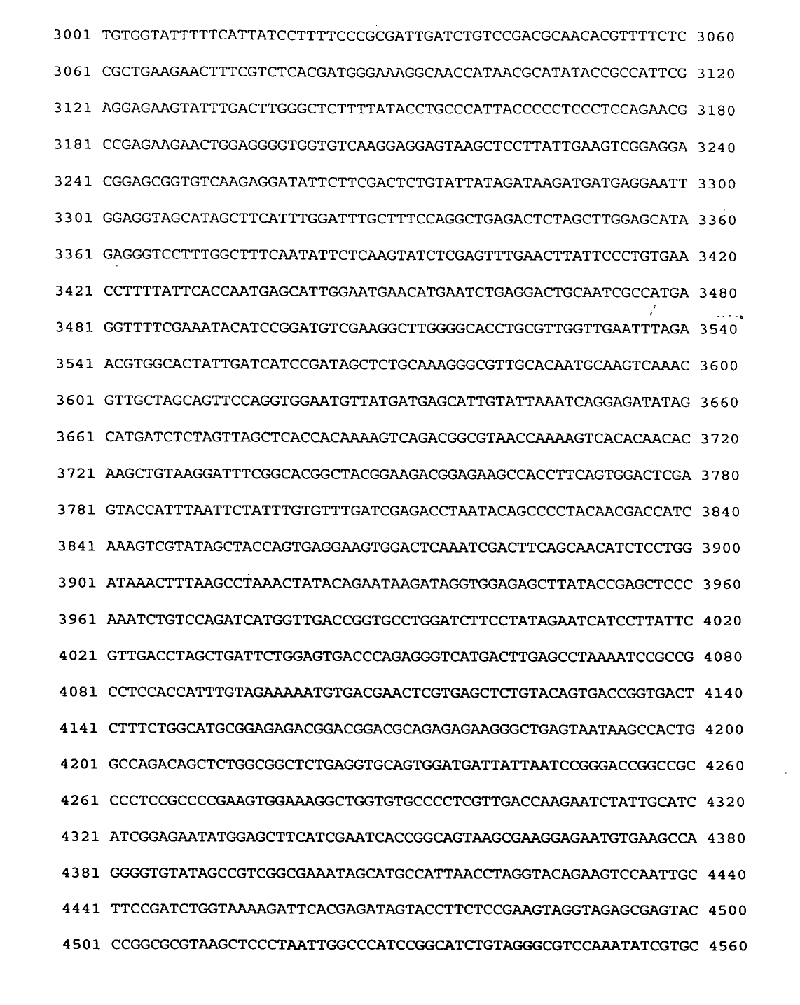

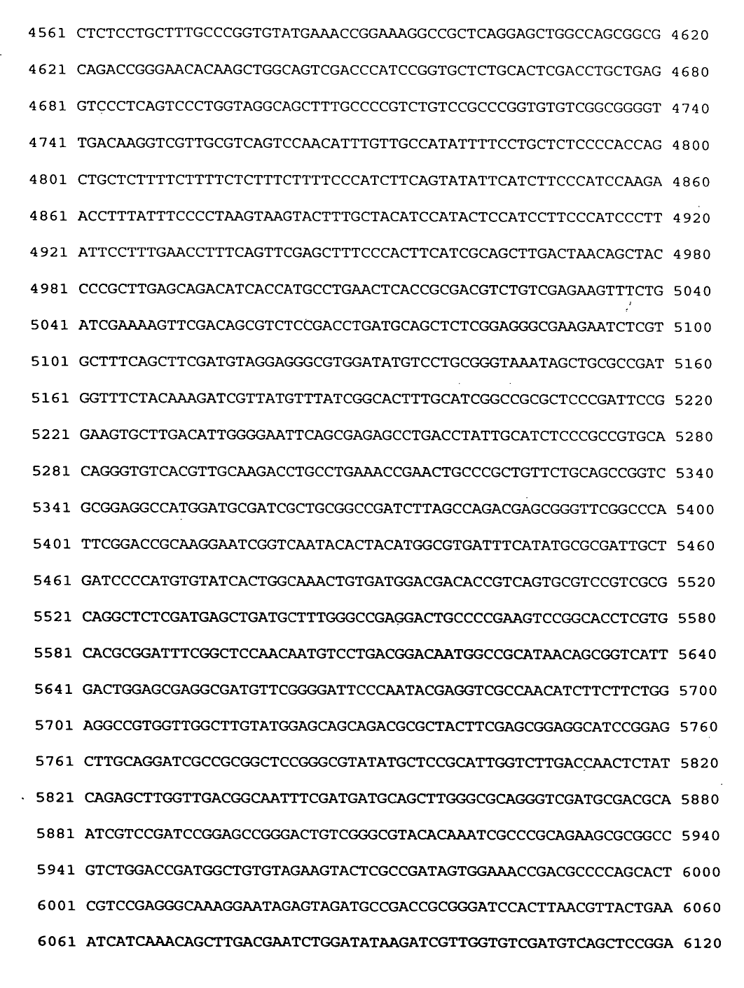

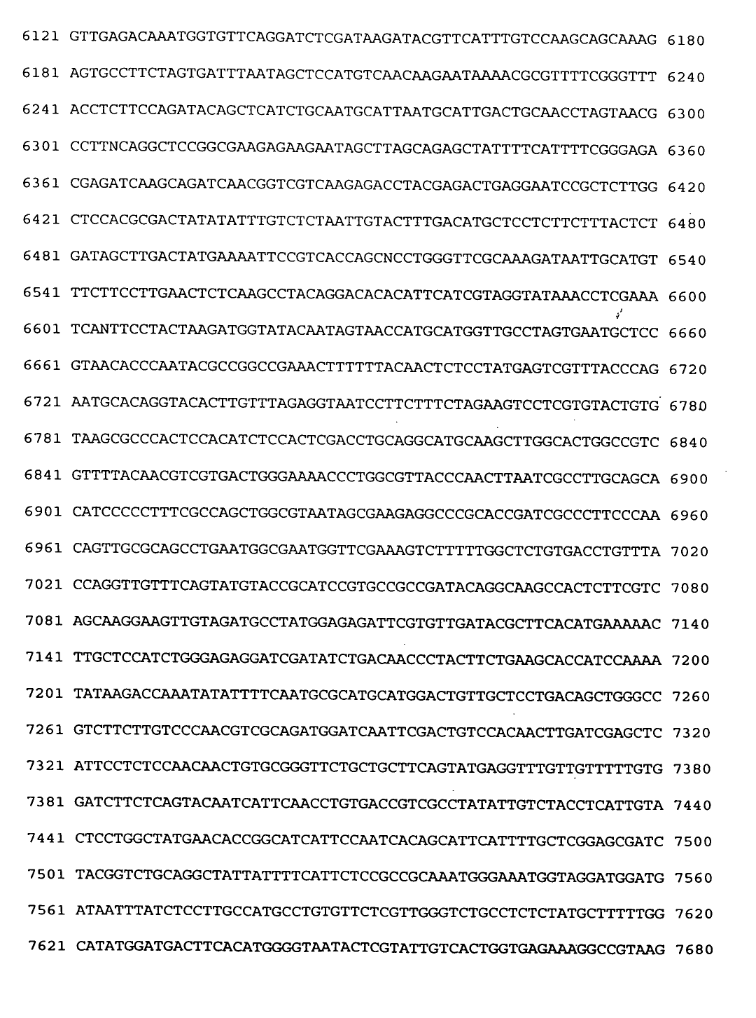

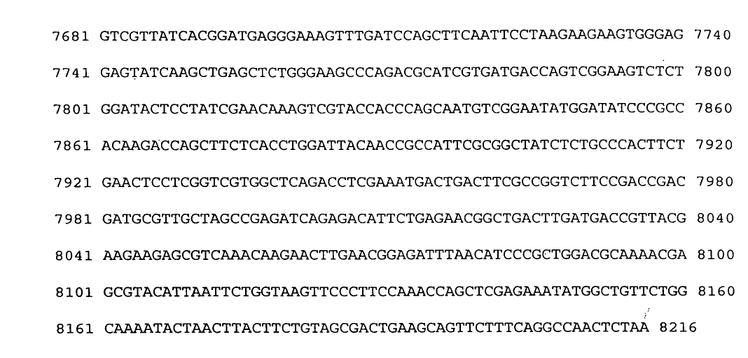

As a representative example, using the methods disclosed herein, a Coccidioides spp. strain was created wherein the resulting transformed CHS5-null mutant had a recombinant CHS5 gene with 8216 nucleotide residues and resulted in no detectable or functional CHS5-encoded polypeptide in transformants. The nucleotide sequence of the recombinant CHS5 gene of the present invention is shown in FIG. 3 (SEQ ID NO:6).

By such methods, additional attenuated strains comprising the introduction of deletions or sequence modifications that would affect target polynucleotide sequences essential to morphogenic conversion and or virulence of Coccidioides spp. are disclosed.

Within the context of the present invention “polynucleotide” in general relates to polyribonucleotides and polydeoxyribonucleotides, it being possible for these to be non-modified RNA or DNA or modified RNA or DNA.

Polynucleotides of the present invention mean the sequences exemplified in this application as well as those which have substantial identity to those sequences and which lead to loss of morphogenic potential of the Coccidioides spp. fungus. Preferably, such polynucleotides are those that hybridize under stringent conditions as defined herein and are at least 70%, preferably at least 80% and more preferably at least 90% to 95% identical to those sequences.

“Consisting essentially of”, in relation to a nucleic acid sequence, is a term used hereinafter for the purposes of the specification and claims to refer to sequences of the present invention and sequences with substitution of nucleotides as related to third base degeneracy. As appreciated by those skilled in the art, because of third base degeneracy, almost every amino acid can be represented by more than one triplet codon in a coding nucleotide sequence. Further, minor base pair changes may result in variation (conservative substitution) in the putative amino acid sequence encoded, are not expected to substantially alter the attenuation or immunologic potential of the fungus. Thus, a nucleic acid sequence as disclosed herein, may be modified slightly in sequence (e.g., substitution of a nucleotide in a triplet codon), and yet still result in the loss of morphogenic conversion and virulence of the fungus because it consists essentially of the same sequence.

The terms “stringent conditions” or “stringent hybridization conditions” includes reference to conditions under which a polynucleotide will hybridize to its target sequence, to a detectably greater degree than other sequences (e.g., at least 2-fold over background). In particular, a DNA or polynucleotide molecule which hybridizes under stringent conditions is preferably at least 70%, 75%, 80%, 85%, 90%, 95%, 97%, 98% or 99% homologous to the DNA that encodes the amino acid sequences described herein. In a preferred embodiment these polynucleotides that hybridize under stringent conditions also encode a protein or peptide which upon administration to a subject provides an immunostimulation sufficient to provide some level of immune protection against Coccidioides spp. as described herein.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes for 15 min each in 0.1×SSC/1% SDS at 60 to 65° C.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Naturally, the present invention also encompasses DNA segments that are complementary, or essentially complementary, to the sequence set forth in SEQ ID NO:2. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment SEQ ID NO:4 under stringent conditions such as those described herein.

The nucleic acid segments of the present invention, regardless of the length of the coding sequence itself, may be combined with other nucleic acid and DNA sequences, such as promoters, polyadenylation signals, additional restriction enzyme sites, multiple cloning sites, other coding segments, and the like, such that their overall length may vary considerably. It is therefore contemplated that a nucleic acid segment or fragment of almost any length may be employed, with the total length preferably being limited by the ease of preparation and use in the intended recombinant protocol.

For example, nucleic acid segments or fragments may be prepared that include a short contiguous stretch identical to or complementary to SEQ ID NO:4, such as about a 3,000, 5,000 or 10,000 bp nucleotide stretch, up to about 20,000 base pairs in length. Nucleic acid and DNA segments with total lengths of about 1,000, about 500, about 200, about 100 and about 50 base pairs in length (including all intermediate lengths) are also contemplated to be useful.

It will be readily understood that “intermediate lengths”, in these contexts, means any length between the quoted ranges, such as 3001, 3002, 3003, 3004, 3005, etc.; including all integers through the 200-500; 500-1,000; 1,000-2,000; 2,000-3,000; 3,000-5,000; 5,000-10,000 ranges, up to and including sequences of about 12,001, 12,002, 13,001, 13,002, 15,001, 20,001 and the like.

It will also be understood that this invention is not limited to the use of the particular plasmid sequence of SEQ ID NO:4 used for the transformation of the Coccidioides spp. fungus. Recombinant vectors and isolated DNA segments may therefore variously include the coding region from SEQ ID NO:4, coding regions bearing selected alterations or modifications in the basic coding region, or they may consist essentially of larger polynucleotide sequences that, when introduced into the Coccidioides spp. fungus, nevertheless result in the loss of genes that control morphogenic potential.

The nucleic acid and DNA segments of the present invention contain essentially equivalent polynucleotide sequences that arise as a consequence of point mutations that occur naturally or through the application of site-directed mutagenesis techniques or through such other techniques that are known to those skilled in the art.

III. Preparation and Formulation of Vaccines.

As described herein, the attenuated Coccidioides spp. strains may be introduced into a mammal by injection or other routes of instillation, in one or more administrations, thereby eliciting an immune response protective against Coccidioides spp. infection. In a further embodiment, the attenuated fungal strains and formulations employing the strains may be admixed in various combinations and or admixed with other known proteins, peptides, or adjuvants which are known or believed to facilitate an immunological response, thereby providing enhanced immunity. In an alternative embodiment, the components of the present invention may be administered separately, i.e., at different time points, which is known or believed to facilitate an immunological response, thereby providing protection against Coccidioides spp. infection. For example, the attenuated strain of the present invention can be combined with one or more additional Coccidioides spp. polypeptides or antigens, such as Ag2/PRA106, Csa, Gel1, Ure, or non-Coccidioides protein antigens or toxoids, such as tetanus toxoid, diphtheria toxoid, cholera toxoid, ovalbumin (OVA), or keyhole limpet haemocyanin (KLH).

The pharmaceutically acceptable carriers which can be used in the present invention include, but are not limited to, an excipient, a stabilizer, a binder, a lubricant, a colorant, a disintegrant, a buffer, an isotonic agent, a preservative, an anesthetic, and the like which are commonly used in a medical field.

Also, the dosage form, such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like, can be used appropriately depending on the administration method and the polypeptides of the present invention can be accordingly formulated. Pharmaceutical formulations are generally known in the art and are described, for example, in Chapter 25.2 of Comprehensive Medicinal Chemistry, Volume 5, Editor Hansch et al, Pergamon Press 1990.

The present invention also provides compositions containing the attenuated strains thereof and one or more suitable adjuvants commonly used in the field of immunology and medicine to enhance the immune response in a subject. Examples of such adjuvants include monophosphoryl lipid A (MPL), a detoxified derivative of the lipopolysaccharide (LPS) moiety of Salmonella Minnesota R595, which has retained immunostimulatory activities and has been shown to promote Th1 responses when co-administered with antigens (see U.S. Pat. No. 4,877,611; Tomai et al., Journal of Biological Response Modifiers. 1987. 6:99-107; Chen et al., Journal of Leukocyte Biology 1991. 49:416-422; Garg & Subbarao. Infection and Immunity. 1992. 60(6):2329-2336; Chase et al., Infection and lmmunity.1986. 53(3):711-712; Masihi et al, Journal of Biological Response Modifiers. 1988. 7:535-539; Fitzgerald, Vaccine 1991. 9:265-272; Bennett et al, Journal of Biological Response Modifiers 1988. 7:65-76; Kovach et al., Journal of Experimental Medicine, 1990.172:77-84; Elliott et al., Journal of Immunology. 1991.10:69-74; Wheeler A. W., Marshall J. S., Ulrich J. T., International Archives of Allergy and Immunology October 2001; 126(2):135-9; and Odean et al., Infection and Immunity 1990. 58(2):427432); MPL derivatives (see U.S. Pat. No. 4,987,237) other general adjuvants (see U.S. Pat. No. 4,877,611); CpG and ISS oligodeoxynucleotides (see U.S. Pat. No. 6,194,388; U.S. Pat. No. 6,207,646; U.S. Pat. No. 6,239,116; U.S. Pat. No. 6,339,068; McCluskie, M. J., and H. L. Davis. Vaccine 2002. 19:413422; Ronaghy A, Prakken B J, Takabayashi K, Firestein G S, Boyle D, Zvailfler N J, Roord S T, Albani S, Carson D A, Raz E. Immunostimulatory DNA sequences influence the course of adjuvant arthritis. Journal of Immunology 2002. 168(1):51-6.; Miconnet et al (2002) 168(3) Journal of Immunology pp 1212-1218; Li et al (2001) Vaccine 20(1-2):148-157; Davis (2000) Developmental Biology 104:165-169; Derek T. O'Hagan, Mary Lee MacKichan, Manmohan Singh, Recent developments in adjuvants for vaccines against infectious diseases, Biomolecular Engineering 18 (3) (2001) pp. 69-85; McCluskie et al (2001) Critical Reviews in Immunology 21(1-3):103-120); trehalose dimycolate (see U.S. Pat. No. 4,579,945); amphipathic and surface active agents, e.g., saponin and derivatives such as QS21 (see U.S. Pat. No. 5,583,112); oligonucleotides (Yamamoto et al, Japanese Journal of Cancer Research, 79:866-873, 1988); detoxified endotoxins (see U.S. Pat. No.4,866,034); detoxified endotoxins combined with other adjuvants (see U.S. Pat. No.4,435,386); combinations with QS-21 (see U.S. Pat. No. 6,146,632); combinations of detoxified endotoxins with trehalose dimycolate and endotoxic glycolipids (see U.S. Pat. No. 4,505,899); combinations of detoxified endotoxins with cell wall skeleton (CWS) or CWS and trehalose dimycolate (see U.S. Pat. Nos. 4,436,727, 4,436,728 and 4,505,900); combinations of just CWS and trehalose dimycolate, without detoxified endotoxins (as described in U.S. Pat. No. 4,520,019); chitosan adjuvants (see U.S. Pat. Nos. 5,912,000; 5,965,144; 5,980,912; Seferian, P. G., and Martinez, M. L. Immune stimulating activity of two new chitosan containing adjuvant formulations (2001) Vaccine. 2000. 19(6):661-8). All of the references cited in this paragraph are incorporated herein by reference.

In another embodiment, various adjuvants, even those that are not commonly used in humans, may be employed in animals where, for example, one desires to subsequently obtain activated T cells or to protect valuable or valued animals from infection due to Coccidioides spp.

IV. Administration of Vaccines

As used herein the subject that would benefit from the administration of the attenuated vaccines and formulations described herein include any mammal that can benefit from protection against Coccidioides spp. infection. In a preferred embodiment, the subject is a human. In a second embodiment, the subject is a domestic animal, including but not limited to dog, cat, horse, bovine (meaning any sex or variety of cattle) or other such domestic animals.

By attenuated vaccine capable of eliciting an immune response in a subject human, including vaccination, the invention covers any strain of Coccidioides spp. incapable of morphogenic conversion to the parasitic, virulent phase but that induces an immune reaction that results in or augments the subject's ability to mount some level of immune protection inhibiting Coccidioides spp. infection. In one embodiment, the Coccidioides spp. is Coccidioides immitis. In another embodiment, the Coccidioides spp. is Coccidioides posadasii.

As used herein, “inhibit”, “inhibiting” or “inhibition” includes any measurable or reproducible reduction in the infectivity of Coccidioides spp. in the subject mammal. “Reduction in infectivity” means the ability of the subject to prevent or limit the spread of Coccidioides spp. fungus in tissues or organs exposed or infected by said fungus. Furthermore, “amelioration”, “protection”, “prevention” and “treatment” mean any measurable or reproducible reduction, prevention, or removal of any of the symptoms associated with Coccidioides spp. infectivity, and particularly, the prevention, or amelioration of Coccidioides spp. infection and resultant pathology itself.

The dosages of the attenuated vaccines used to provide immunostimulation include from about 0.1 μg to about 2000 μg, which includes, 0.5, 1.0, 2.0, 5.0, 10, 20, 30, 40, 50, 60, 70, 80, 90, 100, 150, 200, 250, 300, 350, 400, 450, 500, 600, 800, 1000, 1500, and 1800 μg, inclusive of all ranges and subranges there between. Such amount may be administered as a single dosage or may be administered according to a regimen, including subsequent booster doses, whereby it is effective; e.g., the compositions of the present invention can be administered one time or serially over the course of a period of days, weeks, months and or years.

The compositions of the attenuated vaccines can be administered by any suitable administration method including, but not limited to, injections (subcutaneous, intramuscular, intracutaneous, intravenous, intraperitoneal), oral administration, intranasal administration, inhalation, or other methods of instillation known in the art.

V. Kits.

Also included within the scope of the present invention are kits suitable for providing compositions of the attenuated vaccines. For example, in such a kit one vial can comprise the attenuated fungus of the invention admixed with a pharmaceutically acceptable carrier, either in a aqueous, non-aqueous, or dry state; and a second vial which can carry immunostimulatory agents, and or a suitable diluent for the composition, which will provide the user with the appropriate concentration of fungus to be delivered to the subject. In one embodiment, the kit will contain instructions for using the composition and other components, as included; such instructions can be in the form of printed, electronic, visual, and or audio instructions. The vaccinations will normally be at from two to twelve week intervals, more usually from three to five week intervals. Periodic boosters at intervals of 1-5 years, usually three years, will be desirable to maintain protective levels. The course of the immunization may be followed by assays for activated T cells produced, skin-test reactivity, or other indicators of an immune response to Coccidioides spp.

Having generally described the attenuated strains of Coccidioides spp. useful as vaccines and the methods to create and administer them to elicit protective immune responses, a further understanding can be obtained by reference to certain specific examples that are provided herein for purposes of illustration only, and are not intended to be limiting unless otherwise specified.

### EXAMPLES

### Example 1

### Creation and Characterization of CHS5 Null Mutant of Coccidioides posadasii

Materials and Methods

Culture conditions. C. posadasii (isolate C735) was used for all experimental procedures reported in this study. The saprobic (mycelial) phase of the fungus was grown on glucose-yeast extract agar (GYE; 1% glucose, 0.5% yeast extract, 2% agar) or GYE broth for the production of arthroconidia, the asexual reproductive propagule of the saprobic phase, and mycelia, as required for subsequent procedures and experiments described herein.

To confirm the ability of the transformant to grow at physiologic temperature, the transformed Δchs5 strain was cultured on GYE agar were suspended in 15 ml of 1% PBS containing glass beads. The suspension was shaken vigorously, and the fragmented hyphae were washed twice in PBS and resuspended in PBS. One ml of the suspension was inoculated into 50 ml of the following liquid media: GYE broth, RPMI-1640 with fetal bovine serum (American Type Culture Collection, Manassas, Va.), Brain Heart Infusion broth (BHI; Difco Laboratories, Detroit, Mich.), or Antibiotic Medium 3 (AM3; Becton Dickinson Microbiological Systems, Sparks, Md.). The cultures were then incubated at 37° C. with shaking at 150 rpm and observed after 3 days.

Genome database analysis and gene discovery. The C. posadasii genome sequencing project was initiated in 2001 at The Institute for Genomic Research (TIGR, Rockville, Md.), and involves a whole genome shotgun strategy for determination of >99% of the 29-megabase genome sequence. Genomic libraries of C. posadasii (isolate C735) with inserts of 2-10 kilobases (kb) were constructed in the pUC plasmid (Promega, Madison, Wis.), and sequenced from both ends. Each library contained >6×10⁵ recombinants, and the combined recombinants of three libraries have been estimated to be sufficient for sequence analysis of the entire C. posadasii genome (Kirkland, T. N., and G. T. Cole. 2002. Gene-finding in Coccidioides immitis: searching for immunogenic proteins, p. 247-254. In K. J. Shaw (ed.), Pathogen genomics: impact on human health. Humana Press, Totowa, N.J.). Genomic survey sequences (GSS) have been assembled into unique contigs and incorporated into a public database (available at The Institute for Genomic Research web site at tigr.org). Computational analyses of the partial genome database were performed by application of the basic local alignment search tool (BLAST) (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Research 25:3389-3402). Sequence alignments were conducted using the translated nucleotide sequences of the contigs and the non-redundant protein database available from the National Center for Biotechnology Information (Wheeler, D. L., C. Chappey, A. E. Lash, D. D. Leipe, T. L. Madden, G. D. Schuler, T. A. Tatusova, and B. A. Rapp. 2000. Database resources of the National Center for Biotechnology Information. Nucleic Acids Research 28:10-14). BLASTX matches were selected with Expect (E) values of <10⁻⁴ as previously described (Kirkland, T. N., and G. T. Cole. 2002.).

Construction of transformation plasmid. CHS5 gene disruption plasmid vector, pΔCHS5 (SEQ ID NO:4), was a construct of fragments of fungal transformation vector pAN7-1, CpCHS5 and pCR2.1-TOPO. The vector, pAN7-1 (GenBank accession # Z32698), contains the hygromycin B phosphotransferase gene (HPH) (Punt, P J., Oliver, R P., Dingemanse, M A., Pouwels, P H., and van den Hondel, C A. 1987. Transformation of Aspergillus based on the hygromycin B resistance marker from Escherichia coli. Gene 56: 1.17-124), which confers resistance to the transformation selection marker, hygromycin. A 4236-bp fragment of CHS5 (SEQ ID NO:3) was amplified by PCR using the primers SEQ.ID NO:7 and SEQ ID NO:8 (all primer sequences were synthesized by Integrated Technologies, Inc. Coraville, Iowa) and cloned into 3.9-kb plasmid vector, pCR2.1 TOPO (Invitrogen™ Life Technologies, Inc., San Diego, Calif.) to yield 8.1-kb pCHS5-2.1. A 1420-bp CHS5 fragment was deleted from pCHS5-2.1 by tandem digestion with Sma I and Csp45 I, and a 6.8-kb fragment, which contained the pCR2.1 -TOPO fragment flanked by 1056- and 1759-bp lengths of CHS5, was recovered. The transformation vector, pAN7-1, was cut with Stu I, then with Nar I, and a 3822-bp fragment, which included the gpdA promoter, HPH and trpC terminator sequences, was extracted. The 6.8-kb pCHS5-2.1 and 3822-bp pAN7-1 fragments were then ligated and the product, pΔCHS5, was used to transform Escherichia coli strain TAM1. Prior to C. posadasii transformation, pΔCHS5 was linearized by digestion with Apa I and Spe I at the pCR2.1-TOPO fragment. A 6734-bp pΔCHS5, comprising the 3822-bp pAN7-1 fragment, 1759- and 1056-bp flanks of CHS5-homologous fragments, and 36- and 61-bp fragments of pCR2.1-TOPO, was extracted by ethanol precipitation, suspended in MSC buffer (10 mM MOPS, pH 6.5; 1 M sorbitol and 20 mM CaCl₂) and held for transformation.

Fungal transformation procedures. Transformation of C. posadasii was performed using protoplasts of germinated arthroconidia. Arthroconidia from 8 to 10 agar plates with dense sporulating mycelia were harvested by flooding each plate with 5 ml of GYE medium plus 0.1% Tween 80 (Sigma, St. Louis, Mo.), followed by gentle disruption of the mycelia with a flamed wire inoculation loop. Suspensions of arthroconidia from each plate were pooled in a 50-ml tube, vortexed vigorously, and used to inoculate a 1 -liter flask that contained 150 ml of GYE medium. In a typical experiment, the flask contained approximately 1.0×10⁶ to 3.0×10⁶ arthroconidia per ml of GYE medium. The cell suspension was incubated in a gyratory shaker (100 rpm, 30° C., 12 h) to obtain germinated arthroconidia, resulting in germ tubes that were approximately two to five times the length of the conidia. Germ tube formation was monitored microscopically. The germlings were harvested by centrifugation (2,000×g, 4° C., 10 min) and transferred to a sterile, transparent 50-ml polypropylene tube. To obtain C. posadasii protoplasts, the germlings were digested by a cocktail of lytic enzymes including 75-mg Driselase, 40-mg glucanase and 10 U of chitinase T-1 (InterSpex Products, San Mateo Calif.) in 10 ml of osmotic buffer (50 mM potassium citrate buffer, pH 5.8; 0.6 M KCl). The mixture was incubated at 30° C. with shaking at 50 rpm for 1 h, and the protoplast were harvested in MSC buffer as described earlier (5). The concentration of the protoplast was adjusted to 5.0×10⁷/ml of MSC buffer.

For transformation, about 3 μg of the transforming fragment comprising the sequence of SEQ ID NO:4 was mixed with 100 μL of protoplast suspension (approximately 5×10⁶ protoplasts) in a 1.5-ml microcentrifuge tube and thoroughly mixed with 30 μl of 60% polyethylene glycol 3350 (PEG; Sigma) prepared in MSC buffer. After 30 min of incubation on ice, another 900 μl of 60% PEG was mixed with the sample, followed by incubation for 30 min at room temperature. The protoplasts were pelleted by centrifugation (5,000×g, 4° C., 10 min), and the PEG solution was carefully removed by aspiration. The protoplasts were then suspended in 500 μl of MSC buffer transferred in different volumes (10, 25, 50, and 100 μl) to sterile 2-ml polypropylene tubes. Sterile GYE soft agar (1% glucose, 0.5% yeast extract, 0.7% agar, 1 M sucrose) was prepared as a stock and held molten at 45 to 50° C. An aliquot (1.6 ml) of this soft agar was added to each tube, mixed with the protoplasts, and immediately poured onto prewarmed (37° C.) petri plates (100 by 15 mm), which already contained 16 ml of GYE agar plus 1 M sucrose as an osmotic stabilizer. The plates were incubated at 30° C. for 20 h and then overlaid with another 2.4 ml of GYE soft agar containing 1.5 mg of hygromycin. The final concentration of hygromycin after diffusion into the plates was estimated to be 75 μg/ml of agar. The plates were incubated at 30° C. for an additional 6 days. Typically, saprobic-phase colonies of C. posadasii, which were resistant to hygromycin, were visible after 4 days. Putative transformants were isolated with the tip of a 3-ml disposable pipette (March Biomedical Products, Rochester, N.Y.) and transferred to separate, fresh GYE agar plates, which contained 75 μg of hygromycin/ml. For each transformation experiment, a negative control of C. posadasii protoplasts, treated as described above but in the absence of transforming DNA, was prepared.

Putative transformants were selected in overlay of GYE-sucrose soft agar (1% glucose, 0.5% yeast extract, 0.7% agar, 1M sucrose) containing 75-μg/ml hygromycin, and homokaryotic transformants were isolated by repeated (3×) subculturing of single colonies at 10-days intervals in hygromycin-containing GYE agar plates.

PCR analysis of putative transformants. Total genomic DNA was extracted with CTAB (hexadecyltrimethylammonium bromide) buffer (2% w/v CTAB, Sigma; 100 mM Tris-HCl, pH 8.0; 1.4 M NaCl; 20 mM EDTA, pH 8.0; 0.2% v/v β-mercaptoethanol) in 1.5-ml microtubes. About 1 cm² of mycelia from 10-days-old cultures in GYE agar and 500 μL of sterile, acid-washed glass beads (425-600μ; Sigma) were suspended in 500 μL of CTAB buffer, and the cells were disrupted at high speed (500 rpm for 30 s) in a bead beater. Genomic DNA was extracted twice with 500 μL of chloroform/isoamylalcohol and precipitated with two volumes of cold ethanol following the standard protocol (Sambrook, J., Fritsch, E. F, & Maniatis T. (1989). Molecular cloning: a laboratory manual, 2^{nd} ed. Cold Spring Harbor Laboratory Press, Cold Spring Harbor, N.Y.). Genomic DNA was analyzed by PCR for the homologous integration of fragment of linearized plasmid construct, pΔCHS5 (SEQ.ID NO:4), in CpCHS5 locus. The primer pair used to screen for HPH were the sequences of SEQ.ID NO:9 and SEQ.ID NO:10. Amplification products of the expected sizes were sequenced for verification. The putative transformants were also negatively screened by PCR using the primer pair sequences of SEQ.ID NO:11 and SEQ.ID NO:12, which is derived from the deleted region of CHS5.

Confirmation of mutant identity. The identity of transformants as C. posadasii was confirmed by PCR using the primer sequences of SEQ.ID NO:22 and SEQ.ID NO:23, which are derived from the gene encoding a 19-kDa protein, which is Coccidioides-specific antigen (CSA) (Pan S, Cole G T. 1995. Molecular and biochemical characterization of a Coccidioides immitis-specific antigen. Infection and Immunity 63: 3994-4002). Further confirmation of identity of transformants was done by sequence analysis of intervening internal transcribed spacer regions, ITS1 and ITS2, of 18S, 5.8S and 28S ribosomal RNA genes. PCR of the transformant and wild type genomic DNA was performed using the universal fungal primer sequences of SEQ.ID NO:24 and SEQ.ID NO:25, which were designed from conserved regions of 18S and 28S rDNA, respectively (White, T. J., Bruns, T. D., Lee, S. B., & Taylor, J. W. 1990. Amplification and direct sequencing of fungal ribosomal RNA genes for phylogenetics, p. 315-322. In M. A. Innis, D. H. Gelfand, J. J. Sninsky, & T. J. White (ed.), PCR protocols. A guide to methods and applications. Academic Press, San Diego, Calif.). The sequence of amplification product was determined using the ABI BigDye cycle sequencing kit and samples were electrophoresed on an ABI PRISM™ 310 Genetic Analyzer (Applied Biosystems, Foster City, Calif. Sequences were then aligned using the CLUSTAL W program (Thompson, J. D., Higgins, D. G. and Gibson, T. J. (1994). CLUSTAL W: improving the sensitivity of progressive multiple sequence alignment through sequence weighting, position-specific gap penalties and weight matrix choice. Nucleic Acids Research 22:4673-4680). Preparation of C. posadasii genomic DNA and Southern blot analysis. Homologous integration of pΔCHS5 at the CHS5 locus was confirmed by Southern blot analysis. Genomic DNA samples of parental and transformant strains of C. posadasii were digested in tandem and overnight with Pst I and Kpn I. The digest and digoxigenin-labeled DNA Molecular Weight Marker II (Roche Diagnostics, Indianapolis, Ind.) were separated on 0.8% (w/v) agarose gel by electrophoresis. The DNA was then transferred to Hybond-N⁺ membranes (Amersham Bioscience, Inc., Piscataway, N.J.) by capillary blotting in 20×SSC as described by Sambrook et al. (Sambrook, J., Fritsch, E. F, & Maniatis T. (1989). Molecular cloning: a laboratory manual, 2^{nd} ed. Cold Spring Harbor Laboratory Press, Cold Spring Harbor, N.Y.).

Labeling of PCR-generated hybridization probes, which comprised of the sequences of SEQ.ID NO:13, SEQ.ID NO:16, SEQ.ID NO:17, and SEQ.ID NO:18, was performed with digoxigenin labeling kit (Boeringer Mannheim GmbH, Germany) according to the manufacturer's instruction. Briefly, PCR was performed on denatured (5 min at 94° C.) genomic DNA for 30 cycles, each consisting of 25 s at 94° C., 25 s at 58° C. and 30 s at 72° C. A final elongation step at 72° C. for 5 min completed the reaction. Amplification products of the expected sizes were purified by gel extraction using the Qiaex II gel extraction kit (Qiagen Inc. Valencia Calif.). Digoxigenin (DIG)-labeling was done by PCR in 25-μL reaction mixture containing 0.7 mM DIG-11-dUTP (Boehringer), 1.3 mM dTTP, 2 mM each of dATP, dCTP and dGTP, 10 ng of the PCR-generated DNA fragment, 1×PCR buffer with MgCl₂ (Perkin Elmer Cetus), 0. 12.5 pM of each primer, and 0.75 μl of Taq polymerase. The primer sets used for both the primary PCR and the digoxigenin-labeling reaction comprised the sequences of SEQ.ID NO:9, SEQ.ID NO:10, SEQ.ID NO:11, SEQ.ID NO:12, SEQ.ID NO:14, SEQ.ID NO:15, SEQ.ID NO:19, and SEQ.ID NO:20.

Hybridization was performed according to standard procedures (Sambrook et al. 1989). The membrane was pre-hybridized at 42° C. for 2 h in 10 ml of pre-hybridization solution (50% formamide, 6×SSC, 5× Denhart reagent, 0.5% SDS, and 100 μg Salmon sperm DNA). Hybridization was performed using 50 μL of 1:10 dilution of denatured (100° C., 5 min), DIG-labeled probe in 10 ml of pre-hybridization solution. Blots were probed overnight at 42° C., washed in 0.1×SSC-0.5% SDS at 68° C. for high stringency. Probes were then coupled with Anti-digoxigenin POD, Fab antibody fragment (Roche), and developed with the ECL™ western blotting Detection reagents (Amersham Biosciences) according to the manufacturers' instructions. The blot was exposed to a Kodak Biomax MR (Eastman Kodak Co., Rochester, N.Y.) for 8 min, and developed.

Results

Deletion of CHS5 and confirmation of mutant identity. The linearized, fragment of the plasmid construct, pΔCHS5, was designed to integrate into C. posadasii chromosomal DNA by a double crossover event at the CHS5 locus. Transformation of 10⁶ protoplasts with 3 μg of pΔCHS5 yielded twenty-two hygromycin-resistant colonies. Three morphological phenotypes were selected following the PCR confirmation of the absence of targeted CHS5 region and presence of HPH. The deletion of a 1420-bp CHS5 fragment (SEQ.ID NO:5) in the mutant genome was confirmed by PCR amplification of the genomic DNA isolated from the mutant and wild-type C. posadasii strains followed by separation and detection on 1-% agarose. A 1257-bp fragment consistent with CHS5 was detected from the wild type but not from the mutant. The integration of the transformation construct, pΔCHS5, at the CHS5 locus was confirmed by PCR amplification and detection of a 580-bp HPH fragment from the mutant but not from the parental wild-type genomic DNA. The identity of the transformant was confirmed by the amplification of a 517-bp fragment of CSA, encoding a Coccidioides-specific 19-kDa protein (Pan S, Cole G T. 1995), from the genomic DNA of the mutant and wild type.

Further confirmation of identity of the mutant transformant was done by PCR amplification and sequence alignment analysis of intervening internal transcribed spacer regions, ITS1 and ITS2, of 18S, 5.8S and 28S ribosomal RNA genes. An alignment of nucleotide sequence of the ITS1 and ITS2 of the mutant and the wild type showed 100% identity by use of the CLUSTAL W program.

Southern Analysis of CHS5-Null Mutants.

Restriction maps were constructed of a 7022-kb genomic fragment containing CHS5 and the contiguous 5′ and 3′ flanking regions, and a hypothetical fragment produced as a result of a homologous, double cross over integration of pΔCHS5 at the CHS5 locus. To confirm that integration of pΔCHS5 into the C. posadasii genome, and that the transformant was homokaryotic, genomic DNA of the Δchs5 and the parental strain was extracted, digested with Pst I and Kpn I, and analyzed by Southern hybridization using the appropriate digoxigenin-labeled probes, comprising the sequences of SEQ.ID NO:13, SEQ.ID NO:16, SEQ.ID NO:17 or SEQ.ID NO:18. A gene deletion event by a homologous double crossover event would result in the loss of a 1420-bp CHS5 fragment (SEQ.ID NO:5) and the integration of the 6637-bp Spe I/Apa I pΔCHS5 fragment (nucleotides 37 to 6673 of SEQ.ID NO:4) at the CpCHS5 locus. Following digestion of Δchs5 genomic DNA with Pst I/Kpn I, 5.3- and 1.5-kb fragments should be detected by use of the probe sequence of SEQ.ID NO:17, which was derived from pAN7-1 (due to the presence of a Pst I site in the HPH gene) and loss of 1.4-kb CHS5 fragment containing the Kpn I site; the Pst I/Kpn I-digested parental genomic DNA should not be hybridized. Hybridization with the probe sequence of SEQ.ID NO:18, derived from the 5′ region of the CHS5 outside the pΔCHS5 construct, would also confirm the homologous integration of the construct at a single locus by detecting 5.3- and 3.7-kb restriction fragments on the Δchs5 and parental strain, respectively. The probe sequence of SEQ.ID NO:16 should hybridize only the Pst I/Kpn I-restricted parental genomic DNA to detect 3.7- and 1.4-kb fragments. Use of the probe sequence of SEQ.ID NO:13, derived from the 3′ region of the CHS5 outside the pΔCHS5 construct should detect fragments of equal size in the restricted genomic DNA of the mutant and parental strains. Two of the three phenotypes had undergone a CHS5 gene deletion event by a homologous double crossover event, as confirmed by Southern blot analysis of the digests using the probes described above, and the third was the result of integrations at the CHS5 locus and elsewhere in the genome. These data provide additional evidence that homologous integration had occurred at a single locus, resulting in deletion of the CHS5 gene, and yielded two homokaryotic Δchs5 transformant strains.

Morphology of CHS5-Null Mutants.

The most striking phenotypic feature of the Δchs5 is the lack of the cottony aerial mycelium, which is typical of C. posadasii. The Δchs5 colony was wrinkled at the center, radially furrowed with sparse, short aerial mycelium. The radial growth rates of mutant and wild-type strains were distinguishable by significantly slower growth of the former. On GYE agar, the Δchs5 colony measured an average of 3 cm in diameter after 11 days, and the parental strain measured 6 cm. Microscopic examination revealed that the mutant hyphae was disfigured and failed to produce arthroconidia. The Δchs5 hyphae had balloon-shaped swellings along their length and at the tips, and treatment of hyphae with Blankophor, a fluorochrome that stains chitin, revealed normal septa in the mutant but resulted in intense staining in the swollen regions indicating an accumulation of chitin. The Δchs5, unlike the parental strain, failed to grow and undergo morphogenesis to the parasitic phase of this fungus when cultured in Converse medium at 39° C. However, the Δchs5 strain vigorously grew as hyphae in both defined and complex media incubated at 37° C., with the same disfigured and swollen morphology observed on microscopic examination. No parasitic-phase forms were observed in any of the cultures. Importantly, this confirms the ability of the attenuated strain to grow at physiologic temperature while remaining in the non-parasitic, hyphal phase.

### Example 2

Evaluation of Virulence of CHS5-Null Mutant (Δchs5) in BALB/c Mice.

The virulence of the mutant was assessed in female BALB/c mice at ages 7-8 weeks by two methods. Hyphal fragments of Δchs5 and the wild type C. posadasii were used for infection of mice. Fungi were grown in GYE liquid medium at 30° C. for 1 week. The cultures were harvested by centrifuging at 5000 rpm and 4° C. for 15 min. The mycelium was then suspended in 10 ml of PBS containing 5-mm glass beads, and clumps of mycelium were dispersed by vortexing. The suspension was washed twice in PBS and suspended in PBS. The number of hyphae in the suspension was counted with a haemocytometer, and the colony forming units (CFU) determined by agar plating.

PBS suspensions (4×10³ CFU) of the wild type and Δchs5 mutant strains were injected intraperitoneally separately in either of two groups of ten mice. At one and two weeks post-challenge, injected mice from each group were euthanized and cultured to determine the extent of infection. The liver, spleen, kidneys and connective tissue of the intestines were examined for evidence of pathological lesions, and recoverable CFU were determined by plating homogenized portions on GYE agar medium. Representative portions of the organs were fixed in neutral buffered formaldehyde 10%, embedded in paraffin wax and processed for histopathology. Sections (3 μm in thickness) of the embedded tissues were stained with methenamine silver (Grocott) for light microscopy observations.

Mouse-survival test. In a separate experiment, using methods described above, groups of twelve female BALB/c mice were injected with either the Δchs5 mutant or the wild type C. posadasii strain, and survival of the mice was monitored for 70 days.

Results

In the virulence experiments, pathological lesions were not observed in the organs of euthanized and necropsied mice injected with Δchs5. On histopathologic examination, no fungal propagules characteristic of Coccidioides infection, were found in H&E- or Groccot methenamine silver-stained sections of the liver, spleen or kidney. Cultures of organ homogenates were negative for fungal growth from the mice injected with Δchs5.

In the survival test, there was 100% survival of the mice injected with Δchs5 by 70 days post-challenge, while none of the mice injected with the wild-type parental strain of C. posadasii survived beyond 15 days.

The results confirm that the Δchs5 strain had been attenuated by the complete loss of virulence, compared to wild-type C. posadasii.

### Example 3

Evaluation of Δchs5 as a Vaccine Against Coccidioides posadasii Infection in BALB/c Mice.

Preparation of vaccines. Fungal suspensions, used in the vaccines to be tested, were produced from 7-day old cultures of the Δchs5 strain and wild-type C. posadasii grown in GYE liquid medium. The mycelia were harvested by centrifugation (5000 rpm, 15 min, 4° C.), and suspended in PBS. Hyphae were fragmented by vortexing with 5-mm glass beads. The suspension was allowed to stand for 10 minutes. to precipitate the gross material, and the fine suspension was then transferred to another tube, washed thrice in PBS, and suspended in PBS. Viability was assessed by plating appropriate dilutions on GYE agar, and the CFU determined. Formalin-killed Δchs5 and wild type C. posadasii suspensions were also prepared by suspending a portion of the respective washed hyphal material in 1-% (w/v) paraformaldehyde (Polysciences Inc., Warrington, Pa.) preparation in PBS and incubating for 24 h at RT. The suspensions were then centrifuged, washed four times in PBS and suspended in PBS. The non-viability of the formalin-killed fungus was confirmed by absence of growth in 3-d culture of 100 μL of the suspension on GYE agar. The final preparations, which were used to vaccinate mice, were 50:50 (v/v) mixture of incomplete Freund's adjuvant (IFA) and PBS suspension of Δchs5, formalin-killed Δchs5 or formalin-killed mycelium of wild-type C. posadasii, respectively. The control preparation was a 50:50 (v/v) mixture of PBS and IFA.

Vaccination groups and challenge protocol. Groups of mice, each comprising 8-week-old female BALB/c mice, were used in this study. Separate groups of mice were vaccinated twice, 2 weeks apart subcutaneously at two sites with 100 μL of the appropriate vaccine preparations or the control. Four weeks later, each mouse was infected by the intranasal route with 100 viable arthroconidia of wild-type C. posadasii in a volume of 30 μL (Lawrence R M, Huston A C, & Hoeprich P D. 1977. Reproducible method for induction pulmonary coccidioidomycosis in mice. Journal of Infectious Diseases 135:117-119.). Intranasal administration of arthroconidia was done under light halothane (1,1,1-trifluoro-2,2-chlorobromoethane) anesthesia. Infected mice were monitored for survival for 45 days and surviving mice were analyzed 45 days after infection for extent of lung infection, by plating of homogenized lung on GYE agar and determination of fungal CFU.

Results

In the first vaccination experiment, two groups of 15 mice each were vaccinated with either Δchs5-IFA or PBS-IFA as a negative control, and then challenged with C. posadasii. Surprisingly, the results showed that protection could be conferred in mice against C. posadasii infection by the vaccination of mice with priming and booster doses of 1.3×10³ CFU and 2.4×10³ Δchs5 CFU, respectively. There was a significant difference between the survival of negative control mice and Δchs5-vaccinated mice after the intranasal challenge with wild-type C. posadasii; all Δchs5-vaccinated mice survived beyond the 45 day period of observation, while all the control animals were dead by 26 day after challenge. At the end of the experiment, the surviving mice were necropsied and examined. Localized nodules of healed abscesses were observed in the lungs of the Δchs5-vaccinated mice. Cultures of lung homogenates of nine (60%) of the surviving mice had no detectable CFU, indicating that they had acquired significant immunity to the otherwise lethal infection.

In a second experiment, four groups of 14 mice each were vaccinated with primer and booster doses of 250 CFU of Δchs5, formalin-killed Δchs5 derived from the live Δchs5 preparation, formalin-killed wild-type C. posadasii or PBS. All mice vaccinated with either PBS-IFA negative control or formalin-killed wild type C. posadasii were dead 29 days post-challenge with wild-type C. posadasii. In comparison, 100% of Δchs5-vaccinated and 43% of formalin-killed Δchs5 survived the lethal challenge to the end of the 45-day observation period. Surviving mice were necropsied and examined. Fibrotic abscesses were observed in the lungs of five of the six surviving mice that were vaccinated with formalin-killed Δchs5, and lung homogenates were culturally positive for C. posadasii. All of the Δchs5-vaccinated survivors, with exception of one, had no detectable CFU in their lungs indicating once again the acquisition of a strong immune response.

These surprising results confirm the utility of the attenuated Δchs5 as a vaccine for the prevention of coccidioidomycosis, as evidenced by the complete protection of the vaccinated mice challenged by an otherwise highly lethal challenge.

The following experiments are proposed as additional examples that could be conducted to further illustrate the utility of the invention:

### Example 4

Creation and Characterization of CHS7-Null Mutant of Coccidioides posadasii as an Attenuated Vaccine

Materials and Methods

Culture conditions. C. posadasii (isolate C735) would be used for all experimental procedures in this study. The saprobic (mycelial) phase of the fungus would be grown on glucose-yeast extract agar (GYE; 1% glucose, 0.5% yeast extract, 2% agar) or GYE broth for the production of arthroconidia, the asexual reproductive propagule of the saprobic phase, and mycelia, as required for subsequent procedures and experiments described herein.

Genome database analysis and gene discovery. The C. posadasii genome sequencing project was initiated in 2001 at The Institute for Genomic Research (TIGR, Rockville, Md.), and involves a whole genome shotgun strategy for determination of >99% of the 29-megabase genome sequence. Genomic libraries of C. posadasii (isolate C735) with inserts of 2-10 kilobases (kb) were be constructed in the pUC plasmid (Promega, Madison, Wis.), and sequenced from both ends. Each library contained >6×10⁵ recombinants, and the combined recombinants of three libraries have been estimated to be sufficient for sequence analysis of the entire C. posadasii genome (Kirkland, T. N., and G. T. Cole. 2002. Gene-finding in Coccidioides immitis: searching for immunogenic proteins, (p. 247-254. In K. J. Shaw (ed.), Pathogen genomics: impact on human health. Humana Press, Totowa, N.J.). Genomic survey sequences (GSS) have been assembled into unique contigs and incorporated into a public database (available at The Institute for Genomic Research web site at tigr.org). Computational analyses of the partial genome database were performed by application of the basic local alignment search tool (BLAST) (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Research 25:3389-3402). Sequence alignments were conducted using the translated nucleotide sequences of the contigs and the non-redundant protein database available from the National Center for Biotechnology Information (Wheeler, D. L., C. Chappey, A. E. Lash, D. D. Leipe, T. L. Madden, G. D. Schuler, T. A. Tatusova, and B. A. Rapp. 2000. Database resources of the National Center for Biotechnology Information. Nucleic Acids Research 28:10-14). BLASTX matches would be selected with Expect (E) values of <10⁻⁴ as previously described (Kirkland, T. N., and G. T. Cole. 2002. Gene-finding in Coccidioides immitis: searching for immunogenic proteins, p. 247-254. In K. J. Shaw (ed.), Pathogen genomics: impact on human health. Humana Press, Totowa, N.J.).

Construction of transformation plasmid. The CHS7 gene deletion plasmid vector, pGEM-AN8-CHS7 (SEQ.ID.NO.37), would be a construct of fragments of the fungal transformation vector pAN8-1, CHS7 and pGEM-T Easy (Promega Corp., Madison, Wis.). The vector, pAN8-1 (GenBank accession # Z3271), contains the phleomycin-binding protein gene (BLE) [Punt, P J., Mattern, I E, and van den Hondel, CAMJJ 1988. A vector for Aspergillus transformation conferring phleomycin resistance. Fungal Genetics Newsletter 35, 25-30]. A 3055-bp fragment of CHS7 (SEQ ID NO:26) would be amplified by PCR using the primers SEQ.ID.NO.27 and SEQ ID NO:28 and cloned into 3016-bp plasmid vector, pGEM-T EASY to yield 6071-bp pGEM-CHS7 (SEQ ID.NO.29). PCR of pGEM-CHS7, using the primers SEQ ID NO:31 and SEQ ID NO:32, would yield a 5034-bp fragment, pGEM-CHS7-1037 (SEQ ID NO:30) from which 1037-bp fragment of CHS7 would be deleted. PCR of the transformation vector, pAN8-1, using the primers SEQ ID NO:35 and SEQ ID NO:36, would produce a 3465-bp fragment (SEQ ID NO:34), which would include the gpdA promoter, BLE and trpC terminator sequences. The 5034-bp SEQ ID NO:30 and the 3465-bp pAN8-1 fragments would then be ligated, and the product, 8499- bp pGEM-AN8-CHS7 (SEQ.ID.NO.37), would be used to transform Escherichia coli strain TAM1. Prior to C. posadasii transformation, pGEM-AN8-CHS7 (SEQ ID NO:37) would be linearized by PCR amplification with primers, SEQ ID NO:39 and SEQ ID NO:40, to produce a 5483-bp fragment (SEQ ID NO:38), which would then be used to transform C. posadasii. The linear transformation construct CHS7-pAN8-CHS7 (SEQ.ID.NO.38), comprised of 3465-bp pAN8-1 fragment, 985-bp and 1033-bp flanks of CHS7 fragments, would be designed to effect the deletion of CHS7 at the genomic locus by a homologous, double cross over recombination. The amplification product would be separated on 1-% agarose gel, and the band would be purified, lyophilized and suspended in MSC buffer (10 mM MOPS, pH 6.5; 1 M sorbitol and 20 mM CaCl₂).

Fungal transformation procedures. Transformation of C. posadasii would be performed using protoplasts of germinated arthroconidia. Arthroconidia from 8 to 10 agar plates with dense sporulating mycelia would be harvested by flooding each plate with 5 ml of GYE medium plus 0.1% Tween 80, followed by gentle disruption of the mycelia with a flamed wire inoculation loop. Suspensions of arthroconidia from each plate would be pooled in a 50-ml tube, vortexed vigorously, and used to inoculate a 1-liter flask that contained 150 ml of GYE medium. The cell suspension would be incubated in a gyratory shaker (100 rpm, 30° C., 12 h) to obtain germinated arthroconidia, resulting in germ tubes that were approximately two to five times the length of the conidia. Germ tube formation would be monitored microscopically. The germlings would be harvested by centrifugation (2,000×g, 4° C., 10 min) and transferred to a sterile, transparent 50-ml polypropylene tube. To obtain C. posadasii protoplasts, the germlings would be digested by a cocktail of lytic enzymes including 75-mg Driselase, 40-mg glucanase and 10 U of chitinase T-1 (InterSpex Products, San Mateo Calif.) in 10 ml of osmotic buffer (50 mM potassium citrate buffer, pH 5.8; 0.6 M KCl). The mixture would be incubated at 30° C. with shaking at 50 rpm for 1 h, and the protoplast would be harvested in MSC buffer as described earlier (5). The concentration of the protoplast would be adjusted to 5.0×10⁷/ml of MSC buffer.

For transformation, about 3 μg of transforming fragment would be mixed with 100 μL of protoplast suspension (approximately 5×106 protoplasts) in a 1.5-ml microcentrifuge tube and thoroughly mixed with 30 μl of 60% polyethylene glycol 3350 (PEG) prepared in MSC buffer. After 30 min of incubation on ice, another 900 μl of 60% PEG would be mixed with the sample, followed by incubation for 30 min at room temperature. The protoplasts would then be pelleted by centrifugation (5,000×g, 4° C., 10 min), and the PEG solution would be carefully removed by aspiration. The protoplasts, which had presumably taken up the DNA at this stage, would be suspended in 500 μl of MSC buffer transferred in different volumes (10, 25, 50, and 100 μl) to sterile 2-ml polypropylene tubes. Sterile GYE soft agar (1% glucose, 0.5% yeast extract, 0.7% agar, 1 M sucrose) would be prepared as a stock and held molten at 45 to 50° C. An aliquot (1.6 ml) of this soft agar would be added to each tube, mixed with the protoplasts, and immediately poured onto prewarmed (37° C.) petri plates (100 by 15 mm), which already contained 16 ml of GYE agar plus 1 M sucrose as an osmotic stabilizer. The plates would be incubated at 30° C. for 20 h and then overlaid with another 2.4 ml of GYE soft agar containing 15 μg phleomycin/ml of agar. The plates would be incubated at 30° C. for an additional 6 days. Typically, saprobic-phase colonies of C. posadasii, which are resistant to phleomycin, would be visible after 4 days. Putative transformants would be isolated with the tip of a 3-ml disposable pipette and transferred to separate, fresh GYE agar plates. For each transformation experiment, a negative control of C. posadasii protoplasts, treated as described above but in the absence of transforming DNA, would be prepared. Putative, homokaryotic transformants would be isolated by repeated subculturing of single colonies at 10-day intervals in hygromycin-containing GYE agar plates.

PCR analysis of putative transformants. Total genomic DNA would be extracted with CTAB (hexadecyltrimethylammonium bromide) buffer (2% w/v CTAB, 100 mM Tris-HCl, pH 8.0; 1.4 M NaCl; 20 mM EDTA, pH 8.0; 0.2% v/v β-mercaptoethanol) in 1.5-mi microtubes. About 1 cm² of mycelia from 10-days-old cultures in GYE agar and 500 μL of sterile, acid-washed glass beads (425-600μ) would be suspended in 500 μL of CTAB buffer, and the cells would be disrupted at high speed (500 rpm for 30 s) in a bead beater. Genomic DNA would be extracted twice with 500 μL of chloroform/isoamylalcohol and precipitated with two volumes of cold ethanol following the standard protocol (Sambrook, J., Fritsch, E. F, & Maniatis T. (1989). Molecular cloning: a laboratory manual, 2^{nd} ed. Cold Spring Harbor Laboratory Press, Cold Spring Harbor, N.Y.). Genomic DNA would be analyzed by PCR for the homologous integration of fragment of the linear plasmid construct, SEQ ID NO:38, in the CHS7 locus. The primer pair used to screen for BLE would be SEQ ID NO:51 and SEQ ID NO:52. Amplification products of the expected sizes would be sequenced. The putative transformants would be also screened by PCR for the absence of the deleted CHS7 sequence using the primer pair SEQ ID NO::42 and SEQ ID NO:43.

Confirmation of mutant identity. The identity of transformants as C. posadasii would be confirmed by PCR using the primers, SEQ ID NO:22 and SEQ ID NO:23, which are derived from the gene encoding a 19-kDa protein, which is Coccidioides-specific (Pan S, Cole G T. 1995. Molecular and biochemical characterization of a Coccidioides immitis-specific antigen. Infection and Immunity 63: 3994-4002). Further confirmation of identity of transformants would be done by sequence analysis of intervening internal transcribed spacer regions, ITS1 and ITS2, of 18S, 5.8S and 28S ribosomal RNA genes. PCR of the transformant and wild type genomic DNA would be performed using the universal fungal primers ITS4 (SEQ ID NO:24) and ITS5 (SEQ ID NO:25), which would be designed from conserved regions of 18S and 28S rDNA, respectively (White, T. J., Bruns, T. D., Lee, S. B., & Taylor, J. W. 1990. Amplification and direct sequencing of fungal ribosomal RNA genes for phylogenetics, p. 315-322. In M. A. Innis, D. H. Gelfand, J. J. Sninsky, & T. J. White (ed.), PCR protocols. A guide to methods and applications. Academic Press, San Diego, Calif.). The sequence of amplification product would be determined using the ABI BigDye cycle sequencing kit and samples would be electrophoresed on an ABI PRISM™ 310 Genetic Analyzer. Sequences would then be aligned using the CLUSTAL W program (Thompson, J. D., Higgins, D. G. and Gibson, T. J. (1994). CLUSTAL W: improving the sensitivity of progressive multiple sequence alignment through sequence weighting, position-specific gap penalties and weight matrix choice. Nucleic Acids Research 22:4673-4680).

Preparation of C. posadasii Genomic DNA and Southern Blot Analysis.

Homologous integration of SEQ ID NO:38 at the CHS7 locus would be confirmed by Southern blot analysis. Genomic DNA samples of parental and transformant strains of C. posadasii would be digested in tandem and overnight with Nru I and Eco47 III. The digest and digoxigenin-labeled DNA Molecular Weight Marker II (Roche) would be separated on 0.8% (w/v) agarose gel by electrophoresis. The DNA would then be transferred to Hybond-N⁺ membranes (Amersham) by capillary blotting in 20×SSC as described by Sambrook et al. (Sambrook, J., Fritsch, E. F, & Maniatis T. (1989). Molecular cloning: a laboratory manual, 2^{nd} ed. Cold Spring Harbor Laboratory Press, Cold Spring Harbor, N.Y.).

Labeling of PCR-generated hybridization probes, SEQ.ID.NO: 41, SEQ.ID.NO: 44, SEQ.ID.NO: 47 and SEQ.ID.NO: 50, would be performed with digoxigenin labeling kit. Briefly, PCR would be performed on denatured (5 min at 94° C.) genomic DNA for 30 cycles, each consisting of 25 s at 94° C., 25 s at 58° C. and 30 s at 72° C. A final elongation step at 72° C. for 5 min completed the reaction. Amplification products of the expected sizes would be purified by gel extraction using the Qiaex II gel extraction kit (Qiagen Inc.). Digoxigenin (DIG)-labeling would be done by PCR in 25-μL reaction mixture containing 0.7 mM DIG-11-dUTP (Boehringer), 1.3 mM dTTP, 2 mM each of dATP, dCTP and dGTP, 10 ng of the PCR-generated DNA fragment, 1×PCR buffer with MgCl₂ (Perkin Elmer Cetus), 0. 12.5 pM of each primer, and 0.75 μl of Taq polymerase. The same primer sets were used for the primary PCR and the digoxigenin-labeling reaction: SEQ.ID.NO: 42/43, SEQ.ID.NO: 45/46, SEQ.ID.NO: 48/49, SEQ.ID.NO: 51/52 primer pairs for probes SEQ.ID.NO: 41 SEQ.ID.NO: 44, SEQ.ID.NO: 47, and SEQ.ID.NO: 50, respectively.

Hybridization would be performed according to standard procedures (Sambrook et al. 1989). The membrane would be pre-hybridized at 42° C. for 2 h in 10 ml of pre-hybridization solution (50% formamide, 6×SSC, 5× Denhart reagent, 0.5% SDS, and 100 μg Salmon sperm DNA). Hybridization would be performed using 50 μL of 1:10 dilution of denatured (100° C., 5 min), DIG-labeled probe in 10 ml of pre-hybridization solution. Blots would be probed overnight at 42° C., washed in 0.1×SSC-0.5% SDS at 68° C. for high stringency. Probes would then be coupled with Anti-digoxigenin POD, Fab antibody fragment (Roche), and developed with the ECL™ western blotting Detection reagents (Amersham Biosciences) according to the manufacturers' instructions. The blot would be exposed to a Kodak Biomax MR for 8 min, developed and examined.

Deletion of CHS7 and confirmation of mutant identity. The linearized, fragment of the plasmid construct, SEQ.ID.NO: 38, is designed to integrate into C. posadasii chromosomal DNA by a double crossover event at the CHS7 locus. Transformation of 10⁶ protoplasts with 3 μg of SEQ.ID.NO: 38 would be followed by culture. The plate would be examined for phleomycin-resistant colonies and a morphological phenotype selected following the PCR confirmation of the absence of targeted CHS7 region and presence of BLE. Deletion of a 1037-bp CHS7 fragment corresponding to SEQ.ID.NO: 33 would be confirmed by PCR amplification of the genomic DNA isolated from the mutant and wild-type C. posadasii strains. Following the separation of the PCR product on 1-% agarose gel, a 1037-bp fragment consistent with CHS7 would be expected to be detected from the wild type but not from the mutant. The integration of the transformation construct, SEQ.ID.NO: 38, at the CHS7 locus would be confirmed by PCR amplification and detection of a 500-bp BLE fragment from the mutant but not from the parental wild-type genomic DNA. The identity of a successful transformant would be confirmed by the amplification of a 517-bp fragment of CSA, encoding a Coccidioides-specific 19-kDa protein (Pan S, Cole G T. 1995. Molecular and biochemical characterization of a Coccidioides immitis-specific antigen. Infection and Immunity 63: 3994-4002), from the genomic DNA of the mutant and wild type.

Further confirmation of identity of the mutant transformant would be done by PCR amplification and sequence alignment analysis of intervening internal transcribed spacer regions, ITS1 and ITS2, of 18S, 5.8S and 28S ribosomal RNA genes. An alignment of nucleotide sequence of the ITS1 and ITS2 of the mutant and the wild type showed 100% identity by use of the CLUSTAL W program.

Southern Analysis of CHS7-Null Mutants.

Restriction maps would be constructed of the 6215-bp genomic fragment (GenBank accession #: AF533442) containing the CHS7 gene and the contiguous 5′ and 3′ flanking regions, and a 7926-bp hypothetical fragment produced by a homologous, double-crossover integration of SEQ.ID.NO: 38 at the CHS7 locus (SEQ.ID.NO: 53). To confirm that integration of SEQ.ID.NO: 38 into the C. posadasii genome had occurred and that the transformant was homokaryotic, genomic DNA of the Δchs7 and the parental strain would be extracted, digested with Nru I and Eco47 III, and analyzed by Southern hybridization using the appropriate probe, SEQ.ID.NO: 41, SEQ.ID.NO: 44, SEQ.ID.NO: 47 or SEQ.ID.NO: 50. A gene deletion event by a homologous double crossover event would result in the loss of a 1037-bp CHS7 fragment SEQ.ID.NO: 33, and the integration of the 5483-bp SEQ.ID.NO: 38 fragment at the CHS7 locus. Following digestion of Δchs7 or wild-type C. posadasii genomic DNA with Nru I/Eco47 III, two fragments of 1174 bp and 2752 bp should be detected by probe 1 in the wild type genomic digest; the mutant digest should not be hybridized because of deletion of target CHS7. Probe 2 (SEQ.ID.NO: 44), derived from the 5′ region of the CHS7 outside the cross over region, should hybridize a 2752-bp band in the parental genomic DNA, and a 6354-bp band in the Nru I/Eco47 III-digested mutant DNA. The size shift would be because of the deletion of 1037-bp SEQ.ID.NO: 33 CHS7 fragment, which contained an Eco47 III site, and the integration of the 5834-bp SEQ.ID.NO: 38 fragment. Hybridization with probe 3 (SEQ.ID.NO: 47), derived from the 3′ region of the CHS7 outside the cross over region, would also confirm the homologous integration of SEQ.ID.NO: 38 construct at a single locus by detecting 879- and 789-bp restriction fragments on the Δchs7 and parental genomic digests, respectively; a second restriction fragment of equal size should also be hybridized in both genomic digests. Probe 4, derived from the BLE gene should hybridize a 6354-bp restriction fragment in the Δchs7 genomic digest; the parental DNA should not be hybridized. These data would provide evidence that homologous integration had occurred at a single locus, resulting in deletion of the CHS7 gene, and yield homokaryotic Δchs7 transformants.

Morphology of CHS7-Null Mutant.

The colony of CHS7-null mutant would be examined for phenotypic and changes in the mycelium, e.g., lacking the cottony aerial mycelium which is typical of C. posadasii. Microscopic examination would be performed to show that the mutant hyphae were distinguishable from those of the wild-type fungus and no arthroconidia were present. The Δchs7 would be tested for the expected inability to convert to the parasitic phase of this fungus when grown in Converse medium at 39° C., and would be considered for evaluation as an attenuated vaccine if this was shown to be the case.

Evaluation of Virulence of CHS7-Null Mutant (Δchs7) in BALB/c Mice.

The virulence of the mutant would be assessed in female BALB/c mice at ages 7-8 weeks by two methods. Hyphal fragments of Δchs7 and the wild type C. posadasii would be used for infection of mice. Fungi would be grown in GYE liquid medium at 30° C. for 1 week. The cultures would be harvested by centrifuging at 5000 rpm and 4° C. for 15 min. The mycelium would then be suspended in 10 ml of PBS containing 5-mm glass beads, and clumps of mycelium would be dispersed by vortexing. The suspension would be washed twice in PBS and suspended in PBS. The number of hyphae in the suspension would be counted with a haemocytometer, and the colony forming units (CFU) determined by agar plating.

PBS suspensions (10⁴CFU) of the wild type and Δchs7 mutant strains would be injected intraperitoneally separately in either of two groups of ten mice. Infected mice from each group would be euthanized and cultured one and two weeks post-challenge to determine the extent of infection. The liver, spleen, kidneys and connective tissue of the intestines would be examined for evidence of pathological lesions, and recoverable CFU would be determined by plating homogenized portions on GYE agar medium. Representative portions of the organs would be fixed in neutral buffered formaldehyde 10%, embedded in paraffin wax and processed for histopathology. Sections (3 μm in thickness) of the embedded tissues would be stained with methenamine silver (Grocott) for light microscopy observations and examined for the presence of fungal elements. Mouse-survival test. Using the methods described above, groups of twelve female BALB/c mice would be infected with either the Δchs7 mutant or the wild type C. posadasii strain, and survival of the infected mice would be monitored for 70 days. If there was 100% survival of the mice infected with Δchs7 by 70 days post-challenge, while none of the mice infected with the wild-type parental strain of C. posadasii survived beyond 15-30 days, the experiment would confirm the loss of virulence in the Δchs7 strain.

Evaluation of Δchs7 as a Vaccine Against Coccidioides posadasii Infection in BALB/c Mice.

Preparation of vaccines. Fungal suspensions to be used as vaccines would be produced from 7-day old cultures of fungi grown in GYE liquid medium. The mycelia would be harvested by centrifugation (5000 rpm, 15 min, 4° C.), and suspended in PBS. Hyphae would be fragmented by vortexing with 5-mm glass beads and the fine suspension would be then transferred to another tube, washed thrice in PBS, and suspended in PBS. Viability would be assessed by plating appropriate dilutions on GYE agar, and the CFU determined. The final preparations, which would be used to vaccinate mice, would be 50:50 (v/v) mixture of incomplete Freund's adjuvant (IFA) and PBS suspension of Δchs7. The control preparation would be a 50:50 (v/v) mixture of PBS and IFA.

Vaccination groups and challenge protocol. Groups of mice, each comprising of 8-week-old female BALB/c mice, would be used in this study to test the ability of the Δchs7 strain to serve as a vaccine. Mice would be vaccinated twice, 2 weeks apart subcutaneously at two sites with 100 μL of the appropriate vaccine preparations or the control. Four weeks later, each mouse would be infected by the intranasal route with 100 viable arthroconidia of wild-type C. posadasii in a volume of 30 μL (Lawrence R M, Huston A C, & Hoeprich P D. 1977. Reproducible method for induction pulmonary coccidioidomycosis in mice. Journal of Infectious Diseases 135:117-119.). Intranasal administration of arthroconidia would be done under light halothane (1,1,1-trifluoro-2,2-chlorobromoethane) anesthesia. Infected mice would be monitored for survival or analyzed 45 days after infection for extent of lung infection, by plating of homogenized lung on GYE agar and determination of fungal CFU.

If the vaccination experiment showed increased survival in mice vaccinated with Δchs7 and a reduction in the recovery of viable fungus from the organs of necropsied mice, this would confirm the strain as an attenuated vaccine useful for prevention of coccidioidomycosis.

### Example 5

Creation and Characterization of Attenuated Mutant Strains of Coccidioides posadasii Useful as Vaccines

Materials and Methods

Using the general methods and approaches described in Example 4 and those known in the art, for example, Sambrook et al., Molecular Cloning: A Laboratory Manual, Third Edition, Cold Spring Harbor Laboratory Press, New York (2001), Current Protocols in Molecular Biology, Ausubel et al (eds.), John Wiley & Sons, New York (2001) and the various references cited therein, attenuated mutant strains would be created by transformation of C. posadasii 735 with gene deletion plasmid vectors designed to delete, by double crossover events, polynucleotide sequences of genes essential for regulation of morphogenic potential in Coccidioides spp. The genes for such knock-out strains would be selected from Coccidioides spp. genes encoding one or more of the following proteins: CHS5, CHS7, HSP70, HSP104, HSP82, HSP90, HSP26, beta-glucosidase 3, beta-glucosidase 5, and parasitic phase-specific protein PSP-1. The genetic sequences encoding said Coccidioides spp. proteins would be obtained as disclosed herein from a public database available at The Institute for Genomic Research web site at tigr.org using computational analyses of the partial genome database by application of the basic local alignment search tool (BLAST) (Altschul, S. F., T. L. Madden, A. A. Schäffer, J. Zhang, Z. Zhang, W. Miller, and D. J. Lipman. 1997. Gapped BLAST and PSI-BLAST: a new generation of protein database search programs. Nucleic Acids Research 25:3389-3402).

Additional genes for such knock-out strains would be selected from Coccidioides spp. orthologs of genes known to control cell wall development or morphogenesis in other fungi; for example genes encoding the following proteins: Psu1, a cell wall synthesis protein reported to be essential for cell wall synthesis in fission yeast (GenBank AB009980, Biochem Biophys Res Commun. 262(2): 368-741, 1999); verprolin (Vrp), involved in cytoskeletal organization and cellular growth in Saccharomyces cerevisiae, (GenBank Ref|xp_{—}324261.1, Mol Microbiol. 10(3):585-96, 1993); DigA, a protein in Aspergillus nidulans required for nuclear migration, mitochondrial morphology and polarized growth. (GenBank Ref|np_{—}588498.1, Mol Genet Genomics.266(4):672-685, Epub 2001); FluG, a protein reportedly essential for asexual development of Aspergillus nidulans (GenBank AAC37414.1, Genetics, Vol.158, 1027-1036, 2001); Ras2, a RAS related GTP-binding protein that controls morphogenesis, pheromone response, and pathogenicity in the plant fungal pathogen Ustilago maydis (GenBank AY149917, Eukaryotic Cell 1 (6): 954-966, 2002); HymA, a protein essential for the development of conidiophore in Aspergillus nidulans (GenBank AJ001157, Mol Gen Genet. 260(6):510-21, 1999). The polynucleotide sequences encoding said proteins would be obtained as disclosed from a public database available at the National Center for Biotechnology Information web site at ncbi.nlm.nih.gov using the accession numbers provided herein. The known sequences from the non-Coccidioides fungi would be used to conduct BLAST searches of the Coccidioides posadasii sequence data available at the TIGR database in order to obtain the corresponding Coccidioides ortholog gene sequences. The Coccidioides sequences would then be used to create sequence alignments using the translated nucleotide sequences of the contigs or complete gene sequences and the non-redundant protein database available from the National Center for Biotechnology Information (Wheeler, D. L., C. Chappey, A. E. Lash, D. D. Leipe, T. L. Madden, G. D. Schuler, T. A. Tatusova, and B. A. Rapp. 2000. Database resources of the National Center for Biotechnology Information. Nucleic Acids Research 28:10-14), BLASTX matches would be selected with Expect (E) values of <10⁻⁴ as previously described (Kirkland, T. N., and G. T. Cole. 2002. Gene-finding in Coccidioides immitis: searching for immunogenic proteins, p. 247-254. In K. J. Shaw (ed.), Pathogen genomics: impact on human health. Humana Press, Totowa, N.J.). Once appropriate sequences are derived, using the methods described herein appropriate transforming plasmids would be constructed and the C. posadasii wild-type strain would be transformed and resulting transformants screened to confirm the loss of the target gene and corresponding functional protein product.

Using the methods described herein, the identity and homology of the mutant transformants would be confirmed by PCR, sequence analysis, and Southern blot analysis by the methods described herein and would be subsequently screened to confirm loss of morphogenic potential to the parasitic phase and such strains would be evaluated for confirmation of lack of virulence in the mouse model previously described. Strains demonstrating lack of virulence would be considered attenuated and would be subsequently screened to confirm their immunogenicity in the vaccination mouse model previously described in Example 4. If the vaccination experiment showed increased survival in mice vaccinated with the attenuated strain and challenged with the wild-type strain and a reduction in the recovery of viable fungus from the organs of necropsied mice, this would confirm the strain as an attenuated vaccine useful for prevention of coccidioidomycosis.

1. A replication competent, Coccidioides spp. fungus that is attenuated by the loss of morphogenic potential of the fungus wherein said fungus does not transform into the virulent, parasitic phase.

2. A recombinant, replication competent, Coccidioides spp. fungus that is attenuated by deletion of all or a portion of a gene responsible for the morphogenesis of the fungus into the virulent, parasitic phase wherein said recombinant fungus does not transform into the virulent, parasitic phase.

3. The recombinant, replication competent, Coccidioides spp. fungus of claim 2, wherein said fungus is attenuated by deletion of all or a portion of a gene that does not express functional polypeptide gene products wherein said gene is selected from the group consisting of CHS5, CHS7, HSP70, HSP104, HSP82, HSP90, HSP26, beta-glucosidase 3, beta-glucosidase 5, parasitic phase-specific protein PSP-1, the Coccidioides spp. ortholog of Psu1, the Coccidioides spp. ortholog of Vrp, the Coccidioides spp. ortholog of DigA, the Coccidioides spp. ortholog of FluG, the Coccidioides spp. ortholog of Ras2, and the Coccidioides spp. ortholog of HymA.

4. The recombinant, replication competent, Coccidioides spp. fungus of claim 2, wherein said fungus is does not express functional polypeptide gene products of one or more CHS genes of said recombinant Coccidioides spp. fungus.

5. The recombinant, replication competent, Coccidioides spp. fungus of claim 2, wherein said fungus does not express a polypeptide comprising the sequence of SEQ ID NO:2.

6. The recombinant, replication competent, Coccidioides spp. fungus of claim 2, wherein said fungus is attenuated by a mutation of CHS5 gene by the deletion of one or more polynucleotides from the CHS5 gene thereby rendering said fungus incapable of expressing CHS5-encoded protein.

7. The recombinant, replication competent, Coccidioides spp. fungus of claim 6, wherein said deletion comprises the polynucleotide sequence of SEQ ID NO:5.

8. The recombinant, replication competent, Coccidioides spp. fungus of claim 6, wherein said deletion consists of the polynucleotide sequence of SEQ ID NO:5.

9. The recombinant, replication competent Coccidioides spp. fungus of claim 2 that is attenuated by the replacement of a functional CHS5 gene with a recombinant CHS5 gene comprising the polynucleotide sequence of SEQ ID NO:6.

10. The recombinant, replication competent fungus of claim 2, wherein said Coccidioides spp fungus is Coccidioides posadasii.

11. The recombinant, replication competent fungus of claim 2, wherein said fungus is combined with a pharmaceutically acceptable carrier.

12. An isolated nucleic acid comprising the sequence of SEQ ID NO:6.

13. A method of eliciting an immune response in a mammal, comprising introducing into the mammal the recombinant fungus of claim 2, in an amount sufficient to elicit an immune response.

14. The method of claim 13, wherein said mammal is a human.

15. The method of claim 13, wherein said mammal is a domestic animal selected from the group consisting of dog, cat, horse, and bovine.

16. The method of claim 13, wherein the recombinant fungus is administered to the mammal by injection.

17. The method of claim 13, wherein the recombinant fungus is administered to the mammal by injection of the recombinant fungus on multiple days.

18. The composition of claim 2, further comprising an isolated Coccidioides spp. polypeptide.

19. The composition of claim 18, wherein said composition is combined with a pharmaceutically acceptable carrier.

20. A method of eliciting an immune response in a mammal, comprising introducing into the mammal a composition comprising the composition of claim 18, in an amount sufficient to elicit an immune response.

21. The method of claim 20, wherein the composition is administered to the mammal by injection.

22. The method of claim 20, wherein said recombinant fungus component of said composition is administered to the mammal by injection of the recombinant fungus followed by separate administration of said isolated Coccidioides spp. polypeptide.

23. The recombinant, replication competent Coccidioides spp. fungus of claim 2 that is attenuated by the targeted replacement of a functional CHS7 gene with a recombinant CHS7 gene comprising the polynucleotide sequence of SEQ ID NO:53.
